(19) United States
(12) Patent Application Publication
(10) Pub. No.: US 2025/0166835 A1
(43) Pub. Date: May 22, 2025

(54) Diagnostic System and Method for Disease Risk Prediction

(71) Applicants: Ayis Theseas Pyrros, Hinsdale, IL (US); Themistocles Anagnos, La Grange Park, IL (US)

(72) Inventors: Ayis Theseas Pyrros, Hinsdale, IL (US); Themistocles Anagnos, La Grange Park, IL (US)

(21) Appl. No.: 18/903,930

(22) Filed: Oct. 1, 2024

Related U.S. Application Data
(60) Provisional application No. 63/600,650, filed on Nov. 18, 2023.

(51) Int. Cl. G16H 50/30; G16H 10/60; G16H 40/67; G16H 50/50

(52) U.S. Cl. CPC G16H 50/30; G16H 10/60; G16H 40/67; G16H 50/50

(57) ABSTRACT

A disease prediction system and method detects one or more biometric signal data and is trained to offer predictions, recommendations, and/or diagnosis, disease prediction, treatment or services for one or more patients. The system trains a machine learning algorithm, for example, a neural network and includes: biometric detection device configured to generate biometric signal data of one or more patients; an electronic memory that includes data representing a trained neural network that has been trained to produce biomarker information or information used in diagnosis. Biomarker information is personalized to the individual patient as defined by the biometric signal data to: normalize the biometric signal data with at least one of: smoothing or filtering texture, shading/lighting; create a 3D volumetric mesh and project to generate 2D biometric image; identify a plurality of body morphology feature set data; generate body morphology composition data [BMCD] and biomarkers; predict at least one biomarker.

### CROSS-REFERENCE TO RELATED APPLICATIONS

This application is a non-provisional application which claims priority to U.S. Provisional Application No. 63/600,650 filed on Nov. 18, 2023, entitled “Mobile Device-Enabled 3D Data Analysis for Deep Learning-Based Disease Risk Prediction” and the entire content of each priority application is herein incorporated by reference in their entirety.

### BACKGROUND

### 1. Technical Field

This application relates generally to the field of medical diagnostic equipment and technology, medical instruments, and treatment devices.

### 2. Description of Related Art

Existing approaches for diagnosing and predicting disease comorbidity have typically involved analyzing individual biomarkers or using single factor statistical models to identify potential correlations between limited biometric parameters and a corresponding individual likelihood of developing specific diseases. These approaches often rely on predefined thresholds or rules to determine disease risk, which may not account for the complex interplay of multiple biomarkers and individual variations in body morphology. For example:
U.S. patent application Ser. No. 17/861,347 (published application US 2023/0016569 filed Jul. 11, 2022 entitled “Comorbidity Prediction from Radiology Images” to Koyejo, Oluwasanmi et. al.;
U.S. Pat. No. 8,504,392 issued Aug. 6, 2013 to Saria Suchi et. al.;
U.S. Pat. No. 10,269,114 issued Apr. 23, 2019 to Reicher Murray A;
US patent application US20240090778 (application Ser. No. 18/527,127) filed Dec. 1, 2023 to Hartley;
US patent application US20150161331 (application Ser. No. 14/558,706) filed Dec. 2, 2014;
US patent application US20170083679 (application Ser. No. 14/857,600) filed Sep. 17, 2015 to Feder Et. Al.; and
Pyrros, Ayis; Borstelmann, Stephen, Mantravadi et. Al. “Opportunistic detection of type 2 diabetes using deep learning from frontal chest radiographs”, Nature Communications, Jul. 7, 2023 (https://www.nature.com/articles/s41467-023-39631-x.pdf), all are incorporated by reference in their entirety. However, these devices do not provide an at home or portable comorbidity disease prediction capable of operating on preselected or preprocessed body morphology criteria on a handheld battery operated device. (see the limitations of conventional devices: https://zozofit.com). Further, these approaches lack the ability to personalize predictions to individual patients based on their unique biometric characteristics.

Communication of personally identifiable information and personal medical data is fraught with security concerns. Traditionally systems do not adequately protect this sensitive information from security threats. Additionally, traditional systems do not provide verifiable and trusted sources of medical data. None of these approaches provide a comprehensive user oriented and secure solution that combines the features described in this disclosure.

### BRIEF DESCRIPTION OF THE DRAWINGS

Other objects and advantages of the application may become apparent upon reading the detailed description and upon reference to the accompanying drawings, in which:

FIG. 1 is a diagram illustrating a diagnostic disease prediction system configured to generate biomarker prediction information to predict comorbidities, in accordance with some embodiments;

FIG. 2 is a diagram illustrating the diagnostic disease prediction system and a biomarker model server in accordance with some embodiments; FIG. 28 is a corresponding flowchart to identify and correlate biomarkers (FIG. 38) with diseases;

FIG. 3 is a diagram illustrating a system configured and flowchart FIG. 38 to create and/or improve biomarker models associated with biometric signal data in accordance with some embodiments;

FIG. 4 is a diagram illustrating a smartphone and a data analytics engine configured to predict comorbidities information, in accordance with some embodiments;

FIG. 5 is a flow diagram illustrating a method for predicting comorbidities information, in accordance with some embodiments;

FIG. 6 is a flowchart of an approach for training a machine learning algorithm in accordance with some embodiments;

FIG. 7 is a flowchart of an approach for training a machine learning algorithm to create and/or improve products/services models associated with patients in accordance with some embodiments;

FIG. 8 is a flow diagram illustrating a method for creating and/or improving products/services models;

FIG. 9 is a diagram of a structure of a machine learning algorithm in accordance with some embodiments;

FIGS. 10, 11, 12 and 14 illustrate pre-processing and processing of data according to some embodiments;

FIG. 13 is a block diagram illustrating a diagnostic disease risk prediction service for generating disease prediction information and FIG. 23 is the corresponding flow chart;

FIGS. 15, 16 and 18 illustrate, smartphone data may be obtained from multiple Biometric Detection Devices;

FIG. 17 is a block diagram of the biometric detection device and may be implemented in user equipment;

FIG. 18 is an example of a biometric prediction and communication protocol with and without a central server.

FIGS. 19, 20, 22 and 33 are block diagrams of a trained neural network disease prediction system according to some embodiments;

FIGS. 24, 25, 26 and 30 are block diagrams of a diagnostic disease prediction system with one or more servers in a network;

FIG. 27 is a flowchart for a diagnostic disease prediction business plan according to one embodiment;

FIG. 29 is a flowchart illustrating multiple levels of service;

FIG. 31 is a block diagram of a patient's electronic device according to one embodiment;

FIG. 32 is a block diagram of a diagnostic disease prediction system;

FIG. 34 is a block diagram of a diagnostic neural network disease prediction system with feedback for retraining;

FIG. 35 is a diagram of an exemplary neural network having multiple layers;

FIG. 36 is a flow chart for obtaining training data, applying the training data 3604, determining the error and applying error to the network;

FIGS. 37 and 38 are flowcharts for mapping biomarkers to disease predictions;

FIG. 39 illustrates an exemplary biometric signal data such as CAT or MRI section images and processed image data (for example smoothed, scaled, compressed) although any suitable exemplary images may be processed.

### SUMMARY

While the application is subject to various modifications and alternative forms, specific embodiments thereof are shown by way of example in the drawings and the accompanying detailed description. It should be understood, however, that the drawings and detailed description are not intended to limit the application to the embodiments.

In aspects, the approaches described herein offer highly effective diagnostics, treatments, options, recommendations, predictions, and insights to diagnose, predict or estimate the probability of medical diseases. In one specific example, estimates of the probability of medical diseases and/or predictive recommendations and/or diagnosis are made via a mobile device to patients.

Today, patients have little to no information to diagnose the wide array of diseases that may be avoided with early diagnostic information. For example, even if a patient is diagnosed with diabetes or heart disease, the treatment options are usually invasive due to the late stage of diagnosis. Patients have little or no research information such as early diagnostic information to initiate an early stage of disease prevention. Conventional medical examinations are expensive and limited to standard examination and testing practices that do not identify early stage or predict diseases. The patient may review medical information from web search engine results and sites that might provide vague and inconsistent and incorrect information not relevant to the patient, and even worse harmful. However, the medical information is limited to just a few symptoms of the potentially hundreds or thousands of relevant types of medical information. Patients will have no choice but to seek expensive and limited medical care or forego medical care all together especially in areas where medical care is lacking. Further, Web search engines are biased by the advertisers paying the web search engine operator so even if a patient seeks medical advice, incorrect or irrelevant search results are provided usually suited to the advertiser and not the patient. According to one embodiment, revenue may be generated by displaying the disease prediction information to the patient using a smart phone, personal computer, laptop, or tablet and in response receiving payment.

With the present invention and in some aspects, biometric signal data is preprocessed to a reduced file size yet highly detailed in specific characteristics and then is fed to a data analytics engine to comprehensively review a patient's biometric signal data and determine if the patient is likely or in the future may encounter one or more diseases. Medical treatment and prevention can be established for the patient that is better suited than conventional medical diagnosis and treatment. The data analytics engine is trained by and can review highly tuned, processed and focused yet detailed and extensive biometric signal data. According to one embodiment, the data analytics engine provides optimal prediction of diseases also known as biomarkers. The data analytics engine receives extensive comprehensive biometric signal data from a large data set of patients and thus can diagnose illnesses, predict future diseases and match the optimal disease prevention treatment plan to the patient. This comprehensive approach goes well beyond the typical diagnostics of diseases that often occurs too late for early treatment. Thus, the data analytics engine and the biomarker predictions are superior to conventional medical diagnosis.

Among other advantages these approaches economically with significantly reduced processing requirements to predict a future disease for a patient from a large set of patient data. For example, the present approaches can provide doctors, medical professionals, and patients early, much more accurate and comprehensive preventative care. Patients, insurance companies, doctors and medical providers benefit by avoiding the need to spend extensive time and money on expensive equipment predicting and thus avoid treating a disease in an advanced stage.

Many of the approaches described herein utilize machine learning approaches and structures. It will be appreciated that some or several approaches may also be implemented by using fixed algorithms and are algorithmic in nature. By “fixed algorithms” or by “algorithmic,” it is meant that functions are implemented according to a fixed algorithm and not by a machine learning approach. These fixed algorithms are typically implemented by hard-coded software or computer instructions and no training using test data is needed or required. It will also be appreciated that the approaches described herein may be implemented as combinations of algorithms and machine learning approaches where some functions are implemented algorithmically, and others are implemented according to machine learning approaches according to any suitable combination.

Among other advantages, the size of the image and data files are fully processed and compressed and are relatively small thus allowing processing on portable devices. For example, a 2400×600 pixel JPEG image is about 540 KB file size, about 29% of the original size. Other suitable sizes are a matter of performance tradeoffs. In still other aspects, pre-processing of the operational inputs is performed. In examples, the pre-processing may include organizing, compressing, aggregating, or normalizing the operational inputs before the data is ingested by the data analytics engine. Other examples of pre-processing the data are possible.

A diagnostic disease prediction system and method detects one or more biometric signal data and predicts disease comorbidity of one or more individual patients. The system includes: a biometric detection device configured to generate biometric signal data of one or more patients; an electronic memory that includes data representing a trained neural network that has been trained to produce biomarker information or information used in diagnosis. The training is performed according to the biometric signal data. The biomarker information is personalized to the individual patient as defined by the biometric signal data to: normalize the biometric signal data with at least one of: smoothing or filtering texture, shading and lighting; create a 3D volumetric mesh in response to the smoothed biometric signal data; render at least one view; identify a plurality of body morphology feature set data; generate body morphology composition data [BMCD] associated with the 3D mesh feature set for each 3D mesh data model; identify a plurality of biomarkers associated with the BMCD; projecting the biometric signal data from a 3D image onto a 2D plane to generate 2D biometric image data; generate trained specific weights for each BMCD to predict at least one biomarker. The data analytics engine is coupled to the trained neural network in the electronic memory; wherein the trained neural network is subsequently deployed and the control circuit is configured to subsequently: receive biometric signal data, generate corresponding body morphology composition data for a patient and apply the trained specific weights to the trained neural network; predict at least two biomarkers in response to applying the trained specific weights to the body morphology composition data.

Training a neural network involves several systematic steps, starting from data preparation to model evaluation and deployment. Here's a summary:

### 1. Data Collection and Preparation

Collect Data: Gather a dataset that will be used to train the model. The data should be relevant to the task (e.g., images, text, numerical data).

Preprocess Data: Clean the data, normalize or scale features, and split it into training, validation, and test sets. For image data, this might include resizing or augmenting images; for text, it could involve tokenization. Normalization in image processing refers to techniques used to adjust the pixel intensity values of an image to ensure that they fall within a certain range or distribution. This process enhances image contrast, reduces illumination variation, and standardizes images before feeding them into machine learning or deep learning models.

Feature Engineering: According to one embodiment, extract relevant features from the raw data, though with deep learning, neural networks can often learn features automatically.

### 2. Model Design

Choose Architecture: Decide on the neural network architecture (e.g., a fully connected network, convolutional neural network (CNN), recurrent neural network (RNN), or a transformer-based model). This depends on the type of task (e.g., image classification, time-series prediction, natural language processing).

Layer Configuration: Set up the layers (input, hidden, and output layers). Choose activation functions (e.g., ReLU, Sigmoid), and consider dropout or batch normalization layers to reduce overfitting.

### 3. Define the Loss Function

Select Loss Function: Choose an appropriate loss function based on the task. Common choices include:

Cross-Entropy Loss: For classification tasks.

Mean Squared Error (MSE): For regression tasks.

### 4. Choose an Optimizer

Select an Optimizer: Optimizers adjust the weights of the network based on gradients. Common optimizers include:

Stochastic Gradient Descent (SGD): With or without momentum.

Adam: A popular optimizer that adapts the learning rate during training.

### 5. Initialize Weights

Weights may be initialized appropriately (e.g., He or Xavier initialization) to prevent problems like vanishing or exploding gradients.

### 6. Training the Model

Forward Propagation: Input data is passed through the network, and predictions are made.

Compute Loss: Compare the predictions with the actual targets using the loss function.

Backpropagation: Compute the gradient of the loss with respect to each weight using the chain rule.

Update Weights: The optimizer updates the network weights to minimize the loss.

Iterate through Epochs: Repeat the process of forward propagation, backpropagation, and weight updates for a fixed number of epochs.

### 7. Validation

Validate the Model: After each epoch or batch, evaluate the model on the validation dataset. This helps monitor the model's generalization to unseen data and prevents overfitting by early stopping or adjusting hyperparameters.

Adjust Hyperparameters: Hyperparameters such as learning rate, batch size, and the number of epochs can be fine-tuned during training.

### 8. Testing

Evaluate on Test Data: After training is complete, evaluate the model's performance on the test set to gauge its generalization performance.

Metrics: Record metrics such as accuracy, precision, recall, F1-score (for classification), or RMSE (for regression).

### 9. Model Tuning (Optional)

Hyperparameter Tuning: Use techniques like grid search, random search, or Bayesian optimization to find the best hyperparameters for the model.

Fine-tuning: If pre-trained models are used, the network can be fine-tuned for the specific task.

### 10. Deploy the Model

Once the model performs well on the test set, it can be deployed for use in production environments. This may involve exporting the model in a format like ONNX, TensorFlow, or PyTorch to be integrated into a larger system.

### 11. Monitor and Update

Monitor Performance: After deployment, continuously monitor the model's performance in the real world.

Retraining: Periodically retrain the model with new data to maintain or improve its performance.

### DETAILED DESCRIPTION

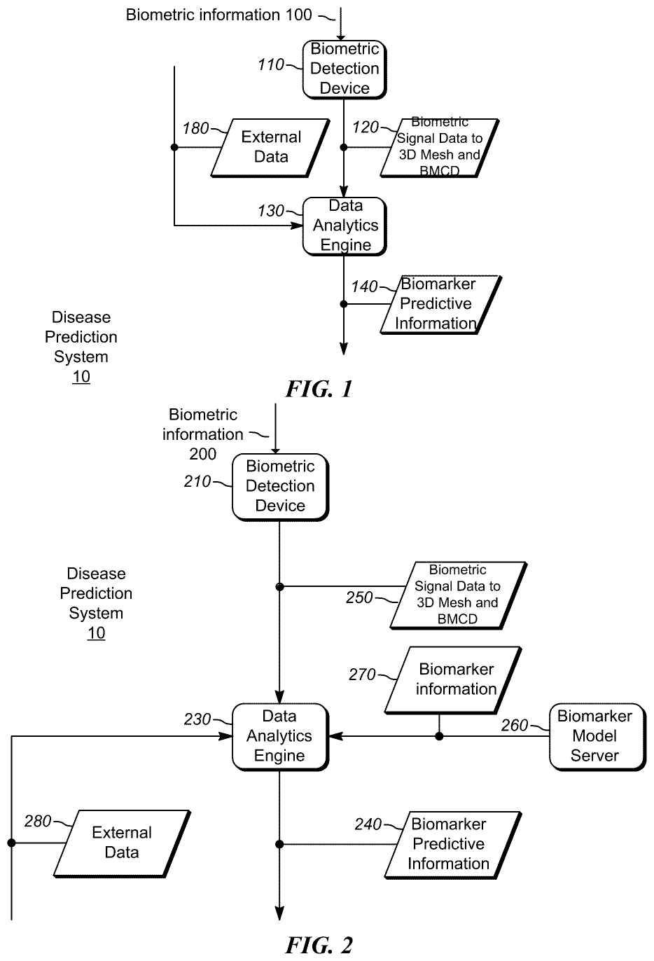

FIG. 1 is a diagram illustrating a diagnostic disease prediction system 10 configured to predict diseases and or provide treatment in response to generating biomarker prediction information 140, 240, 340 associated with patients, in accordance with some embodiments.

The various approaches use biometric information 100, 200 to develop data models, biometric detection device data, and/or external data 180. A data analytics engine 130, 230, 330 may be utilized to make predictions, recommendations, or insights based upon training data, data models, biometric signal data, and/or external data from a large data set of patient biometric data and diagnostic outcomes. The biometric signal data 120, 250, 350 from a patient is data according to at least one of: 3D image, 2D image, lidar, X-ray, MRI, CAT, medical history, EKG, CPAP information and medical test results or any suitable information. Combinations of these elements are also utilized.

In some embodiments, biometric detection device 110 receives biometric information 100 and is configured to generate biometric signal data 120 suitable for further processing and then input to the data analytics engine 130 to generate biomarker predictive information 140. Biometric detection device 110 generates biometric signal data 120 and may associated with one or more patient smartphone-related products/services. Biometric signal data 120 may be provided to data analytics engine 130 for processing. In some embodiments, data analytics engine 130 may be configured to generate biomarker predictive information 140 that is associated with biometric signal data 120. The biomarker predictive information 140 is personalized to the individual patient as defined by the biometric signal data 120.

Biometric signal data 120, 350 may include data from biometric detection device 110, 210, 310 of a large number of patients. The biometric detection device 310 may be for example, light and imaging detectors 110, 210, 310 such as radar, LIDAR sensors, cameras, ultrasonic sensors, MRI, X-Ray, CAT, mobile phone camera, and environmental sensors. Other examples are possible such as sleep study information, CPAP data, heart monitors, ECGs, brain wave monitors, smart health monitors such as Apple and Android smart watches or any suitable biometric detection device.

The data analytics engine 130, 230, 330, 902, 1510 is further configured to receive one or more operational inputs from the sensors, including a smartphone camera from the patient, or from an external source and applying the one or more operational inputs to the trained neural network 1302 (FIG. 13). The output yields insight or prediction(s) from the trained neural network 1302 to predict disease comorbidity and/or diagnosis. The inputs may also be from other test or diagnostic information as described.

In some examples, the data analytics engine 130, 230, 330 may be configured to generate predictive information 140, 240, 340 that is associated with a patient's condition, disease, diagnosis for treatment and/or the one or more products/services. The data analytics engine 130, 230, 330, 902, 1510 is coupled to the sensors and the neural network 1302. It should be noted that the biometric signal data 120, 350 and body morphology feature set data 190 may be received from multiple patients and merged together by the data analytics engine 130, 230, 330. The biometric detection device(s) 110, 210, 310, 916, 1503, 1505, 1507 may be deployed at a smartphone and may be configured to obtain biometric signal data 120 processed to maintain and even enhance biomarkers and biometric data while significantly reducing the data and or file size. The file size roughly depends on these 3 factors: Pixel Size, Compression settings, and Content (detail and complexity of the image).

For example, a typical JPEG file size is about 1 bit per pixel. The degree of compression and file size will vary depending on image content, because certain types of graphics (e.g. flat areas and smooth gradients) compress more than others (noise, texture, shadowing, text), so it's not necessarily a constant compression ratio to apply to every image. A 2400×600 pixel JPEG image is about 540 KB file size, about 29% of the original size. Other suitable sizes are a matter of performance tradeoffs. The smartphone may be operated by a patient, and the first data describing conditions of biometric information 100 of the patient. A compression preprocessing example follows:

Pick a maximum JPEG quality setting based on similar images (somewhere in 70 to 85 range).

Recompress images to that quality level.

If the recompressed image is smaller by more than ˜10%, then keep the recompressed image.

It's important not to choose a merely smaller file size, and require a significant drop in file size instead. That's because recompression of JPEG tends to reduce the file size slightly due to loss of detail caused by lossy nature of JPEG and conversion to Aug. 16, 1932-bit RGB, so small reductions in file size can have disproportionally large drop in quality that does not provide an effective tradeoff between quality and file size. In one example of about one bit per pixel as a guide to filter out 30,000 images out of 100,000+ and re-compress them resulted in an image compression application with 85% quality. If the resulting image was more than 50% smaller then keep the new one.

Biometric signal data 120, 350 may include data from biometric detection device 110, 210, 310 of a large number of patients for example training as further described. The biometric detection device 310 may be for example, light and imaging detectors 110, 210, 310 such as radar, LIDAR sensors, cameras, infrared, ultrasonic sensors, MRI, X-Ray, CAT, mobile phone camera, and environmental sensors or any suitable imaging detector. Other examples are possible such as sleep study information, CPAP data, heart monitors, ECGs, brain wave monitors, smart health monitors such as Apple and Android smart watches or any suitable biometric detection device.

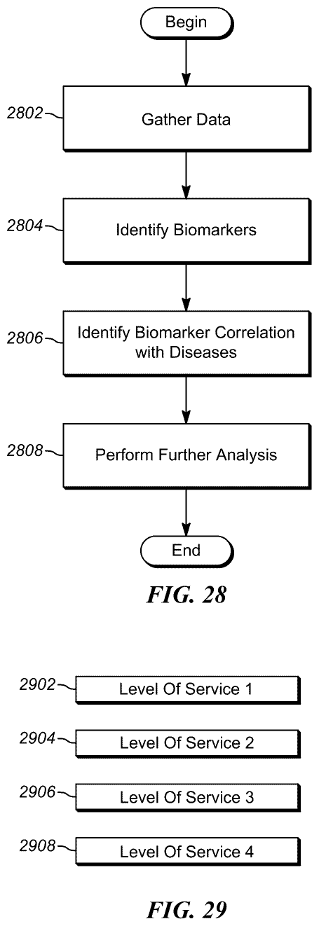

As shown in FIG. 2, products/services based on data models may include data structures that include biomarker information 200. Biomarker information 200 may include, for example, patient conditions, comorbidities, biomarkers, ICD 9 or ICD 10 codes (international classification of disease)], heart disease, diabetes, obesity or any suitable diagnosis and or treatments. A biomarker model server 260 generates and provides biomarker information 270. FIG. 28 is a corresponding flowchart to identify biomarkers and correlate with diseases beginning at step 2802 by gathering relevant biomarker data correlating with diseases at 2806. Biometric signal data 250 and biomarker information 270 is sent to the data analytics engine 230. Further analysis may be performed at step 2808 according to any of the following. It should be noted that biomarker information 270 and biometric signal data 120 may be received from multiple biometric detection devices 110 such as smartphones operated by patients and merged together by data analytics engine 130, 230. FIG. 29 is a flowchart illustrating multiple levels of service.

The present approaches enable highly effective artificial intelligence (AI) powered patient specific biomarkers and may utilize an extensive database of patient information, like radiological databases of Xray, MRI, CAT, heart, brain, sleep, blood tests and the like. New technology knowledge is created based on extensive biomarker information 200. This further reduces cost and helps those economically disadvantaged.

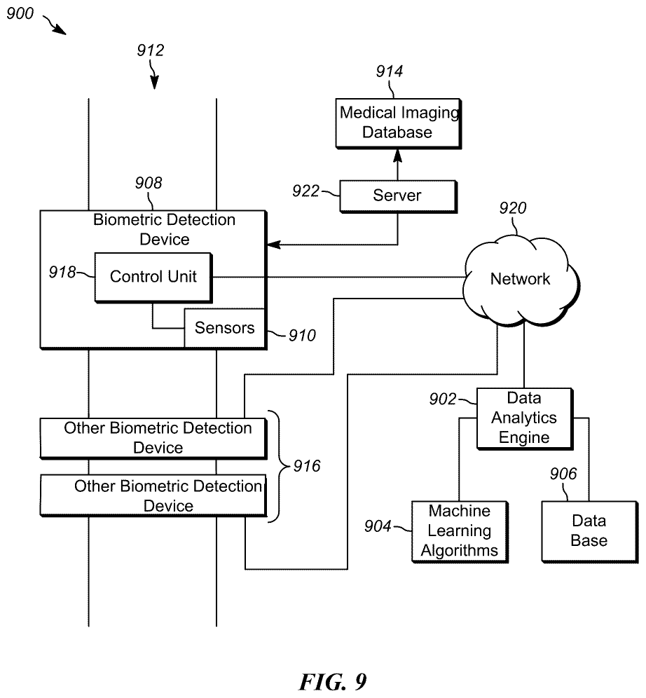

As shown in FIG. 9, the biometric detection device 908 is any type of smartphone such as an mobile terminal, a digital camera, a telematics equipped vehicle, 3D camera, LIDAR, radar, multiple angle and position camera, body scanner, or any suitable imagine device to mention a few examples.

In some embodiments, external data 180, 280 may also be provided to data analytics engine 130. External data 180 may be related to the one or more radiological sources such as Xray, MRI and CAT and may be data comprising a large database of images, medical test result information, patient and diagnostic information received from one or more external sources other than the smartphone(s).

Generally, biometric signal data 120, 250 may include data from one or more sensors on a smartphone, as well as other smartphone data. In addition, smartphone data may include general information about the patient or patients of a specific smartphone. For example, smartphone data may include information identifying the current patient, the patient's sex, age, and other personal information that may influence or identify a patient's health and medical condition(s), and such data may be associated with operational data of the smartphone 110.

In some embodiments, data analytics engine 130, 230 may be implemented using various methods. Data analytics engine 130 may utilize, for example, simple curve-fitting methods, neural network 1302, forms of artificial intelligence, including machine learning, algorithms, large language and language prediction models etc., and combinations of these approaches among other things.

In one embodiment, the data analytics engine 130, 230 may be implemented as an artificial neural network or a parallel distributed processing system. The artificial neural network may include a plurality of interconnected nodes or neurons. Each node of the interconnected nodes may be a node specialized to perform a particular task given one or more inputs.

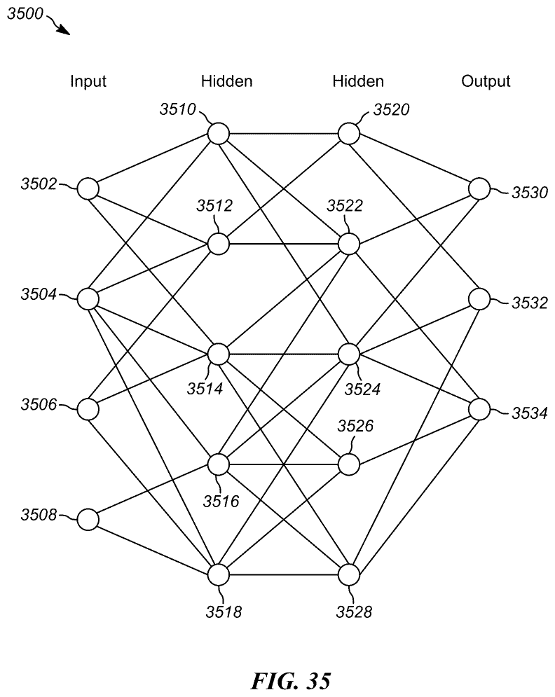

The artificial neural network may include one or more layers of nodes, as shown in one embodiment in FIG. 35. Each layer may include a plurality of nodes, which may be connected to nodes of a previous layer that provide inputs via connections to the nodes of the layer. Additionally, each node within a layer may be configured to generate an output, which may be provided as an input to one or more nodes of a subsequent layer. In this regard, each layer of the artificial neural network may be partially connected or fully connected to one or more other layers of the artificial neural network.

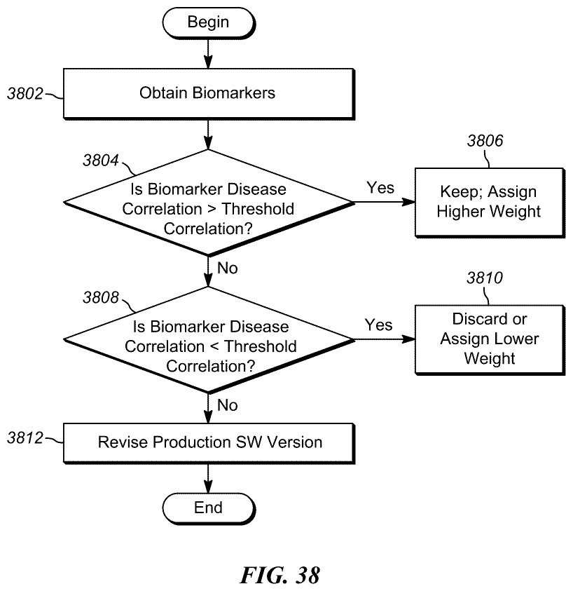

Each connection or input of the one or more inputs to a node may be associated with a weight, as shown in one embodiment in FIG. 38. The weight may represent a relative importance of the input to the node for performing the task of the node. The weights of the inputs or connections to the node may be recursively, iteratively adapted, or optimized based on repetitive operation of the artificial neural network, such that a predictive output of each node and the artificial neural network may be improved. In this regard, the artificial neural network may be trained and retrained according to supervised learning, unsupervised learning, or reinforcement learning.

In some embodiments, biomarker predictive information 140, 240, 340 may include any information that may be learned and predicted by data analytics engine 130 when data analytics engine 130 is provided with biometric signal data 120 and/or external data 180.

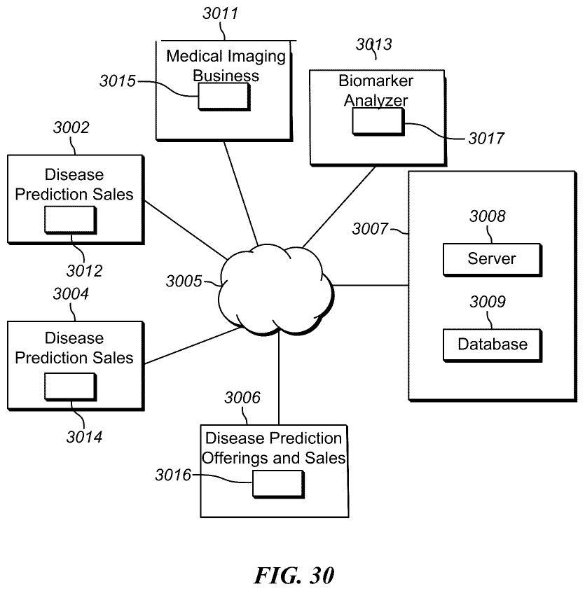

FIG. 21 and FIG. 30 are block diagrams illustrating a service for generating disease prediction information and FIG. 23 is the corresponding flow chart. It will be appreciated that the approaches described herein can also be applied to digital advertising 2302-2312 or other revenue generating service 1312. For example, the output of the machine learning algorithms 904 (e.g., a neural network) can be used to create a customized service 2302 to provide disease prediction information based personalized recommendations for a specific patient. Disease prediction information can be created by the control circuit 902 and pushed to the device 922 via the network 920 to inform the patient of comorbidly predictions 2312. As such disease prediction services may be offered and advertised accordingly.

A neural network 1302 (or other machine learning algorithm or approach) is trained based upon for example, first biometric signal data, second biometric signal data, and/or third biometric signal data as described in FIGS. 13, 15 and 18. The trained neural network 1302 makes predictions or recommendations or offers insights concerning one or more of biomarkers in response to applying the trained specific weights 1308 to the body morphology composition data [BMCD] 1306.

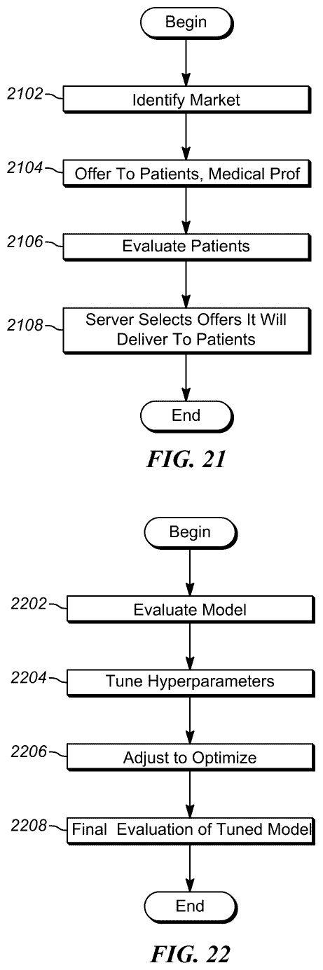

The training of the neural network 1302 is accomplished by differently weighting the first body morphology composition data, the second BMCD, and the third BMCD and so forth (FIGS. 22, 33, 34 and 36).

Subsequently, the trained neural network 1302 is deployed in production. Subsequent to the deployment, one or more operational inputs are received from the biometric detection device, from a large database of patients, and/or from an external source such as radiological data bases and other databases as described such as heart, brain, pancreas, organ and blood tests and relevant data. The one or more biometric detection device inputs are applied to the trained neural network 1302. The processing of the trained neural network 1302 yields an insight, diagnosis, recommendation, or prediction of comorbidity of one or more patients.

A data analytics engine 470 coupled to the trained neural network 1302 in the electronic memory 485, 1706 determines an action based upon the insight or prediction. The action is one or more of: the data analytics engine 470 to receive biometric signal data 120, 350, generate corresponding body morphology composition data 1306 for a patient and apply the trained specific weights to the trained neural network 1302 and predict at least two biomarkers (Biomarker Predictive Information 340) in response to applying the trained specific weights 1308 to the body morphology composition data 1306.

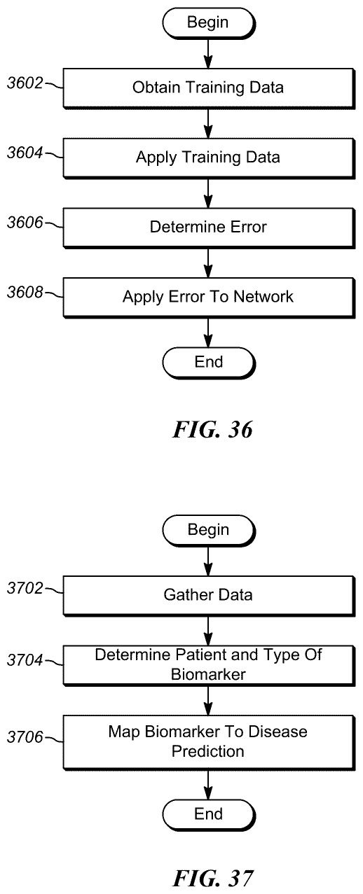

In aspects and after the deployment of the trained neural network 1302, the trained neural network 1302 may be refined, retrained, or restructured to reflect the continued changes to the biometric information 100 and patterns of the patient. Thus, the trained neural network 1302 is retrained to reflect the continued changes to the as more biometric data is received and trains the neural network 1302 (FIGS. 34 and 36).

In examples, the biometric signal data 120, 350, biomarker information 200, 370 and body morphology composition data 1306 are rendered to the patient using a smart phone, personal computer, laptop, or tablet.

In other aspects, the neural network 1302 is deployed at a central location. Other examples of deployment locations (or combinations of different deployment locations such as on a mobile phone, a doctor's office and/or at a central location such as a server) are possible.

In aspects the trained neural network 1302 is created and further improved by training a neural network, the training comprising: receiving first data from the sensors of a biometric detection device 110, 210, 310 such as a smartphone, the smartphone being operated by a patient, the data describing conditions of components of and specifying an patient's health via the biometric information 100 of the patient; receiving second data from other patients, the second data describing biometric information 100 of the other patients; receiving third data concerning biometric information 100 of the patient such as a database of images.

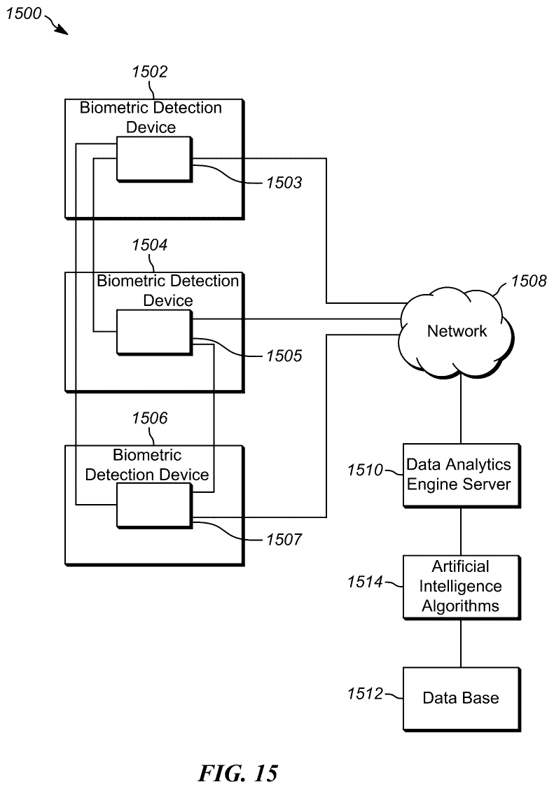

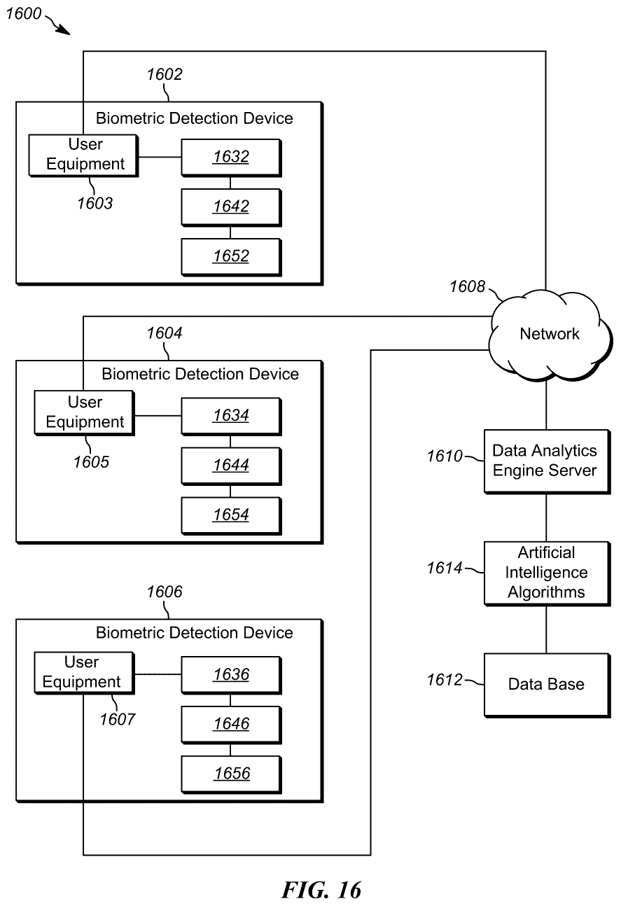

FIGS. 15 and 16 illustrate, smartphone data 1603, 1605 may be obtained from multiple Biometric Detection Devices 1502, 1504, 1506, and in one embodiment as well as from multiple patients. In such embodiments, data analytics engine 130, 1510, 1610 may be configured to combine/correlate the data from the multiple smartphones and patients. In other embodiments, a system for comorbidity prediction includes a plurality of biometric detection device or sensors 916 (FIG. 9, FIG. 18) 1503, 1505, 1507 (FIG. 15), a neural network 1302 (or other machine learning algorithm or approach), and a data analytics engine 130, 230, 330. 902, 1510.

In others of these embodiments, a 2614 server includes a biometric detection device in a smartphone, a neural network 1302 (or other machine learning algorithm), and a control circuit (FIG. 18, 26). The neural network 1302 has been trained with training data sets (FIG. 22).

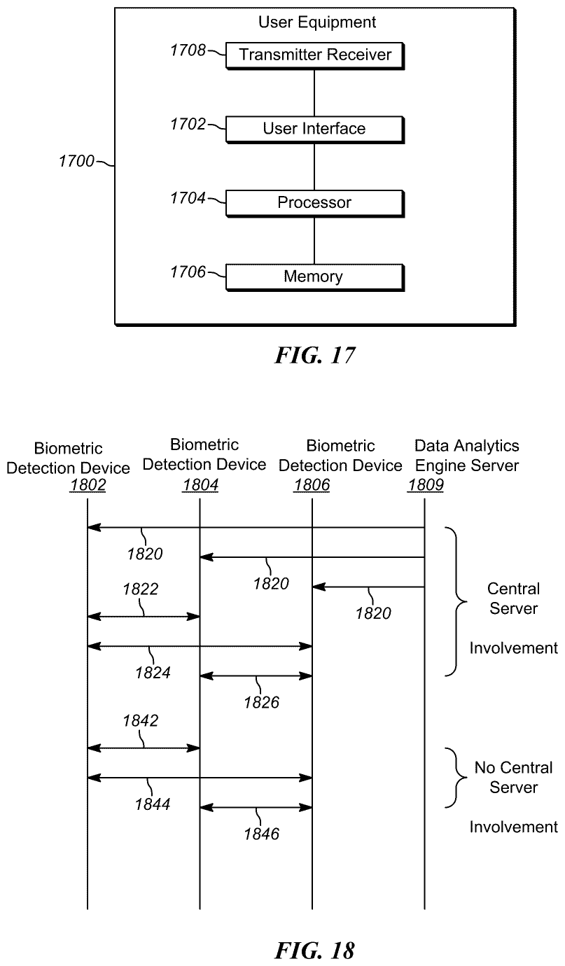

As shown in FIG. 17, the biometric detection device 1704 may be implemented in user equipment 1100. User equipment 1700 includes suitable control unit 918 to control sensors such as an imaging device and to process the images as described with reference to FIG. 9. According to another embodiment, the user equipment 1700 may include a transmitter 1708 for communicating with external devices and (local, co-located, remote) servers using radio waves, light, infrared, wires or any suitable communication medium, a user interface 1702 to allow a user to operate functions such as the imaging device or sensor(s) 1502, 1504, 1506, a processor 1704 and memory 1706. Other examples of smartphones are possible.

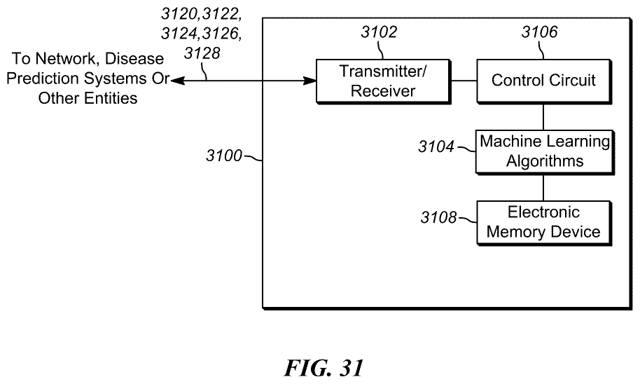

In yet other examples, the neural network 1302 is deployed in a smartphone (FIGS. 17, 31). In still other examples, the disease prediction system 10 includes user equipment for processing such as a smartphone, tablet, watch, medical monitor or device, medical test equipment or other mobile phone device.

FIG. 18 is an example of a biometric prediction and communication protocol with and without a central server and are described throughout. In yet other examples, the disease prediction system 10 is implemented at least partially virtually (FIG. 9, FIG. 30). In still other examples, the neural network 1302 is trained according to a trial-and-error approach.

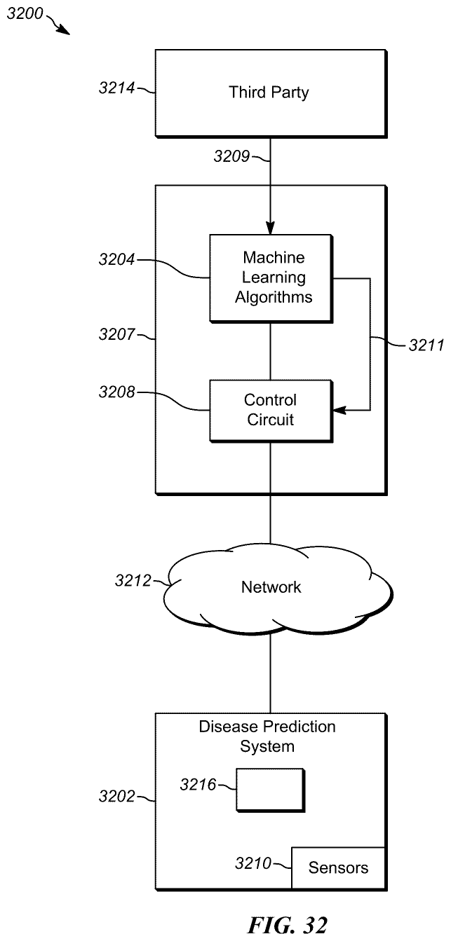

FIG. 32 is a block diagram of a disease prediction system 3202 having a sensor 3210, connected to a network 3212, for communication to an analytics engine node 3207. The analytics engine node 3207 includes a machine learning algorithm 3204 and a control circuit 3208. According to this embodiment, a third party 3214 administers and operates the analytics engine node 3207 and provides predictions and recommendations to one or more disease prediction systems 3202. These embodiments function in a manner similar to the Siri server model where processing is performed in the cloud.

FIG. 21 and FIG. 30 are block diagrams illustrating a service for generating disease prediction information and FIG. 23 is the corresponding flow chart. It will be appreciated that the approaches described herein can also be applied to digital medicine 2302-2312 or other revenue generating services 1312. For example, the output of the machine learning algorithms 904 (e.g., a neural network) can be used to create a customized service 2302 to provide disease prediction information based personalized recommendations for a specific patient. Disease prediction information can be created by the control circuit 902 and pushed to the device 922 via the network 920 to inform the patient of comorbidly predictions 2312.

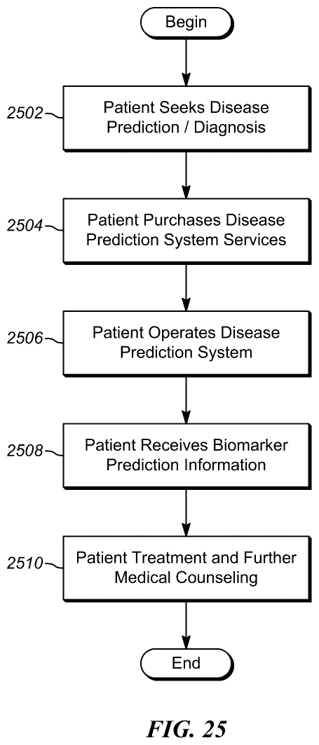

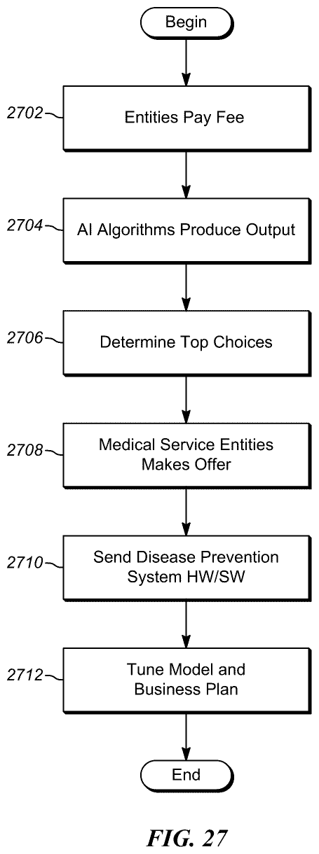

With reference to FIG. 23, FIG. 25, FIG. 27 at step 2302, 2502 a patient purchases services at 2504, 2702 and the data analytics engine 130 may be configured to recommend specific medical service providers at step 2310, 2510, 2706, based at least in part on the provided information at 2306, 2704. For example, if data analytics engine 130 determines that near term or immediate medical attention is needed/appropriate at 2508, a suitable medical specialist may be recommended at step 2310, 2510. Alternatively, if the smartphone requires only routine physical, such as a blood test and vaccinations may be recommended. In some embodiments, certain recommendations/predictive information may be given higher ranking based on other reasons at 2510.

FIG. 32 is a block diagram of a disease prediction system 3202 having a sensor 3210, connected to a network 3212, for communication to an analytics engine node 3207. The analytics engine node 3207 includes a machine learning algorithm 3204 and a control circuit 3208. According to this embodiment, a third party 3214 administers and operates the analytics engine node 3207 and provides predictions and recommendations to one or more disease prediction systems 3202. These embodiment functions in a manner to the Siri server model where processing is performed in the cloud.

Generally, biometric signal data 250 may include data from sensors on the smartphone as well as other smartphone data (such as environmental data, smartphone history, etc.). Biometric signal data 250 may generally include examples such as those discussed in relation to other figures herein.

In embodiments where multiple patients share a smartphone, each set of the smartphone data may be associated with the patient's identity and be used during the determination of the predictive information.

In some embodiments, biomarker model server 260 may be configured to provide data analytics engine 230 with biomarker information 270. Data analytics engine 230 is configured to combine biomarker information 270 with biomarker signal data 250 and optional external data 280 in determining biomarker predictive information 240. In some embodiments, biomarker information 270 may comprise collected and/or processed information about patients and diseases that are being predicted by data analytics engine 230 and are related to the collected smartphone data 250.

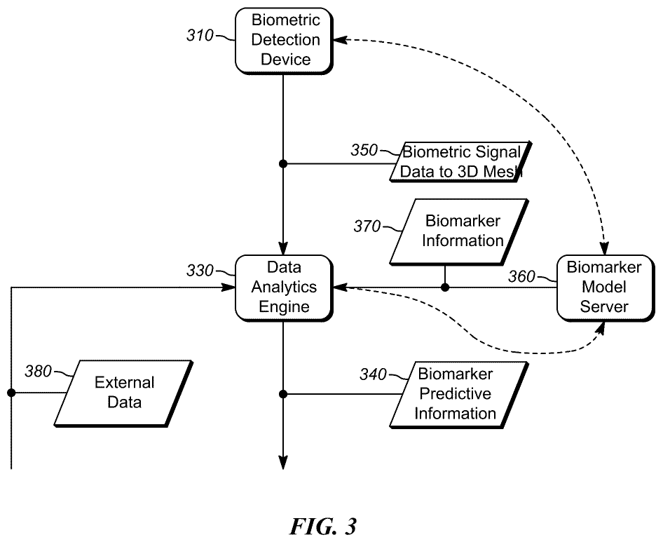

FIG. 3 is a diagram illustrating a system configured to create and/or improve biomarker models associated with biometric signal data for disease prediction and treatment associated with one or more smartphone applications, in accordance with some embodiments.

In some embodiments, biometric detection device 310 is configured to collect biometric signal data 350, which contains information associated with one or more patients. Biometric signal data 350 may then be provided to data analytics engine 330 for processing. In some embodiments, data analytics engine 330 may be configured to generate predictive information 340 that is associated with biometric signal 350.

Generally, biometric signal 350 may include data collected from one or more sensors on a smartphone such as a camera, lidar or similar sensors. Smartphone data may generally include examples as those discussed in relation to other figures here.

In some embodiments, data analytics engine 330 may be implemented using various methods. Data analytics engine 330 may utilize simple curve-fitting methods, neural networks, artificial intelligence methods, etc.

In some embodiments, some pre-processing of the data may occur in order to distill the data to a smaller size prior to transmission. For example, there may be smartphone data obtained from two different biometric detection devices 310 that contains the same or very similar information. In such a case, only data from one of the biometric detection devices may be sent. Additional types of pre-processing, such as general compression, may also be performed locally on the smartphone.

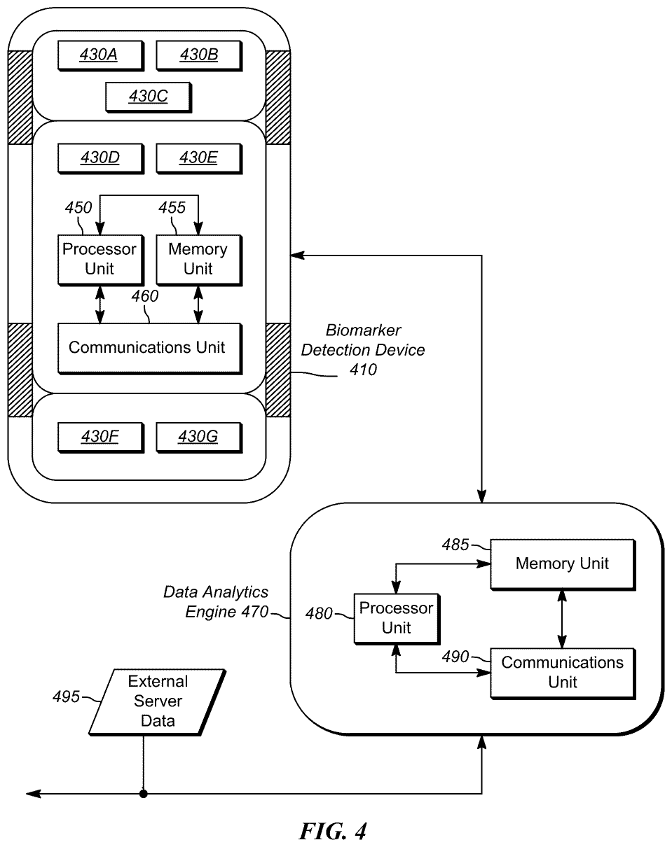

FIG. 4 is a diagram illustrating a biomarker detection device 410 such as a smartphone and a data analytics engine 470 configured to predict comorbidities information, in accordance with some embodiments. Biomarker detection device 410 includes external sensors 430A, 430B, 430C, internal sensors 430D, 430E such as cameras, lidar, processor 450, memory 455, and communications unit 460. In some embodiments, data analytics engine 470 may be configured to apply various methods in generating predictive information. Data analytics engine 470 includes processor 480, memory 485, and communications unit 490. For example, data analytics engine 470 may utilize simple curve-fitting methods, neural networks, artificial intelligence methods, etc.

In some embodiments, data analytics engine 470 may be configured to store, generate, and/or update products/services models described in more detail below. In some embodiments, data analytics engine 470 may be configured to combine products/services models with the smartphone data and the external data 495 in determining the predictive information. In some embodiments, the products/services models may comprise collected and/or processed information about products and services that are being predicted by data analytics engine 470 and are related to the smartphone data. In some embodiments, data analytics engine 470 may be configured to improve the products/services models using the smartphone data and other external data provided to the data analytics engine 470.

Generally, the biomarker predictive information 240 may include information related to products/services that can then be provided to the biomarker detection device 410 and patient. The biomarker predictive information 240 may be sent back to the biomarker detection device 410 or the information may be sent to a designated email address, phone number, doctor's office, medical service provider, insurance company, etc.

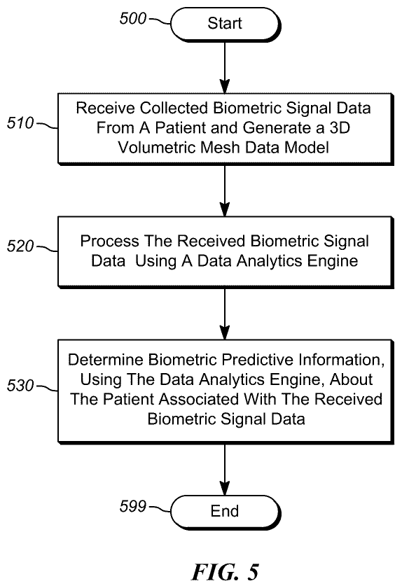

FIG. 5 is a flow diagram illustrating a method for predicting information associated with smartphone products/services, in accordance with some embodiments.

Processing begins at 500 where, at block 510, collected biometric signal data 120 from a biometric detection device 110, 210, 310 such as a smartphone is received. In some embodiments, the collected data may be related to one or more patients associated with a biometric detection device 110, 210, 310.

At block 520 (and 630), the received processed data is processed using a data analytics engine 130, 230, 330. In some embodiments, data analytics engine 130, 230, 330 may be implemented using various methods. Data analytics engine 130, 230, 330 may utilize simple curve-fitting methods, neural networks, and artificial intelligence methods.

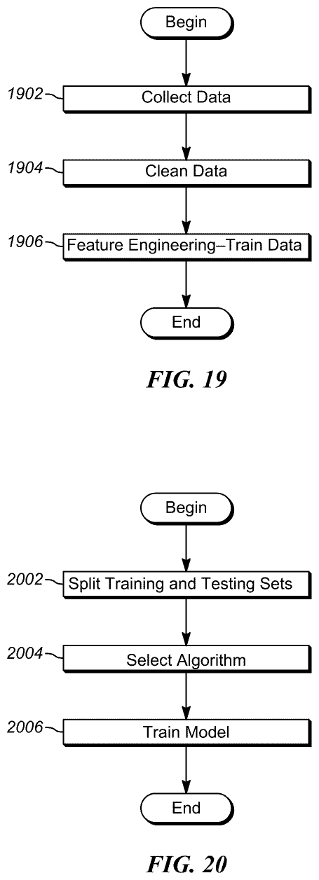

FIGS. 19, 20, 22 and 33 are block diagrams of a trained neural network disease prediction system according to some embodiments.

As shown in steps 1902, 1904, 1906 of FIG. 19, training data is generated according to the desired feature engineering in response to receiving collected biometric signal data 120 from a biometric detection device 110, 210, 310 such as a smartphone after cleaning the data of artifacts as described.

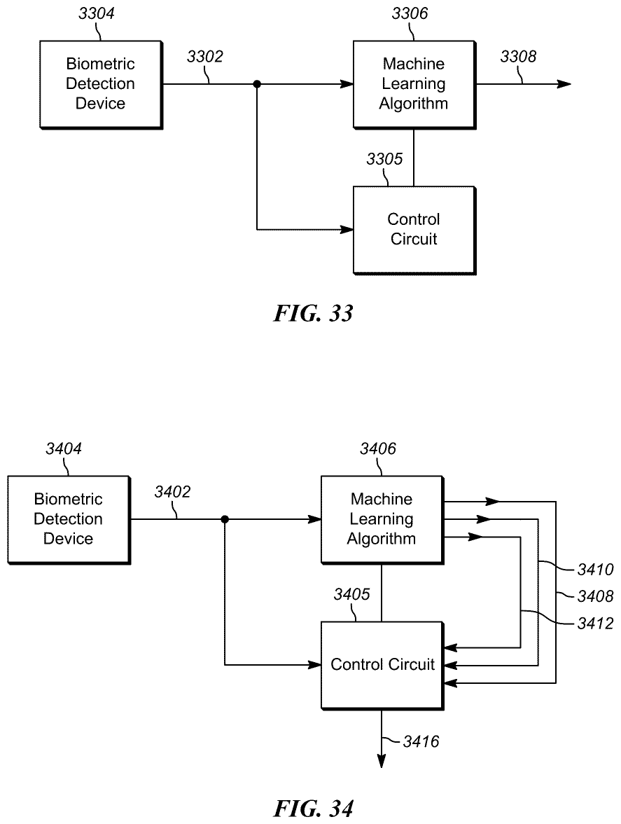

FIG. 33 illustrates a biometric detection device 3304, control circuit 3305 and machine learning algorithm 3306. As described elsewhere herein and when a neural network 1302 is used, the neural network 1302 may be trained for usage using training data. The training data trains the machine learning algorithm 3306. To generate appropriate disease prediction information 3308.

FIG. 34 is a block diagram of a neural network disease prediction system with integrated feedback for retraining.

Once trained, the neural network 1302 can be further refined as new data 3408, 3410, 3212 is received or as specifications change 3402 (to mention two examples). In examples, the neural network 1302 can periodically be retrained or refined, but in other examples the retraining is asynchronous in time and, as such, may be triggered by asynchronous events such as the arrival of new testing data.

At block 530 (640), the data analytics engine 130, 230, 330 determines predictive information 3308.

Processing subsequently ends at 599.

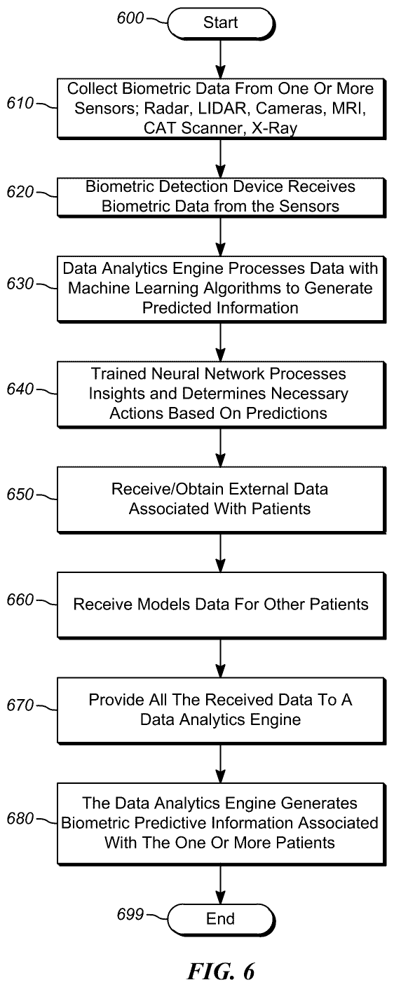

FIG. 6 is a flow diagram illustrating a method for predicting information associated with patients, in accordance with some embodiments.

Processing begins at 600 where, at block 610, data is collected from one or more sensors such as camera, radar, IR, LIDAR, MRI, CAT, infrared, and X-ray on a smartphone. The smartphone data is associated with one or more smartphone-related products/services.

At block 620, the collected smartphone data is distilled. In some embodiments, the smartphone data is reduced in size to better facilitate the transmission of the data. For example, duplicate data may be removed. Generally, normalization, smoothing, filtering such as compression of the data may be performed.

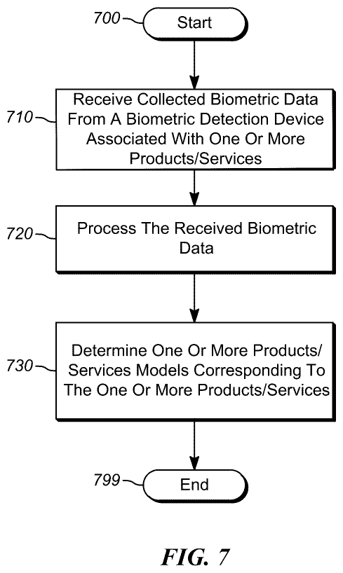

FIG. 7 is a flow diagram illustrating a method for training a machine learning algorithm to create and/or improve products/services models associated with patients, in accordance with some embodiments.

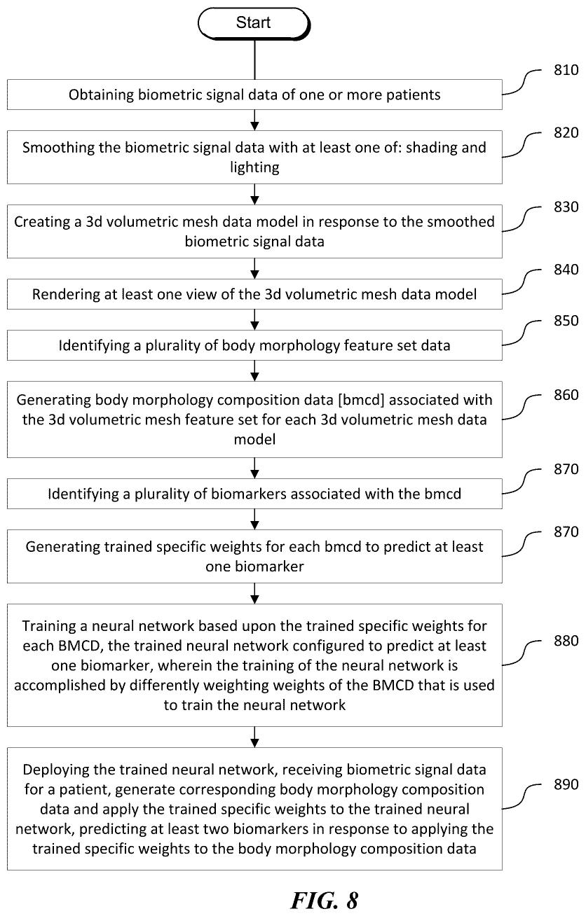

FIG. 8 is a flow diagram illustrating a method for creating and/or improving products/services models associated with smartphone products/services, in accordance with some embodiments. Reference may also be made to FIG. 9. FIG. 9 is a diagram of a structure of a machine learning algorithm in accordance with some embodiments.

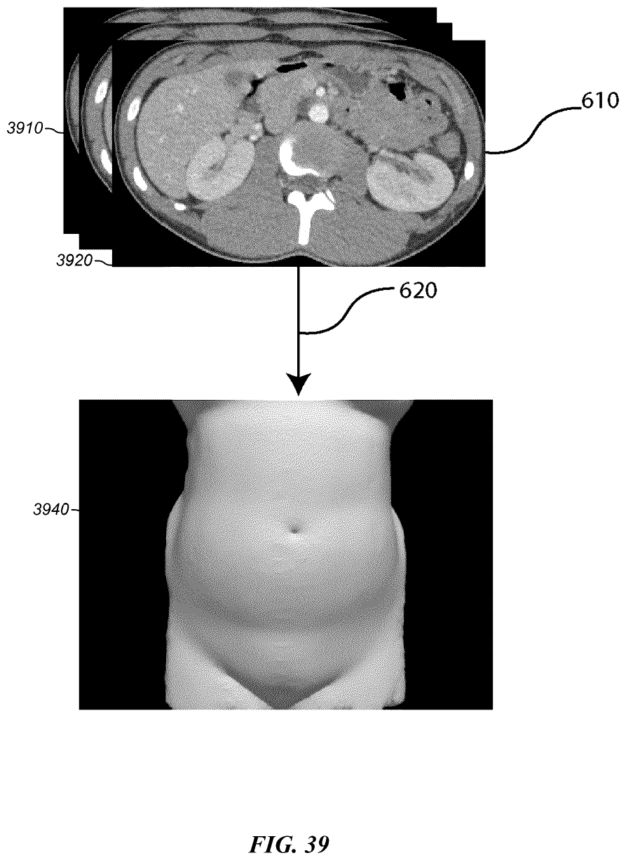

In some aspects, the techniques described herein relate to a method for disease comorbidity prediction and treatment, the method includes at step 810: obtaining biometric signal data 120 of one or more patients. FIG. 39 illustrates an exemplary biometric signal data 120 such as CAT or MRI section images 3910, 610 and processed image data 620, 3940 (for example smoothed, scaled, compressed) although any suitable images may be processed. At step 820 smoothing the biometric signal data 120 with at least one of: shading and lighting. At step 830 creating a 3D volumetric mesh in response to the smoothed biometric signal data 120. According to one embodiment 2D images may be analyzed by projecting the biometric signal data 120 from a 3D image onto a 2D plane to generate 2D biometric image data. Among other advantages, the 2D image requires less processing and is well suited for portable device applications. At step 840 render at least one view. According to another embodiment smoothing or filtering the biometric signal data 120 generates textured data. FIG. 39 illustrates an exemplary rendered view. Further, coloring the biometric signal data 120 generates colored data 190 and shadowing may be performed on the 2D biometric signal data 120. At step 850 identifying a plurality of body morphology feature set data 190. At step 860 generating body morphology composition data 1306 [BMCD] associated with the 3D (or 2D) mesh feature set for each 3D (or 2D) mesh data model. At step 870 identifying a plurality of biomarkers associated with the BMCD; and generating trained specific weights for each BMCD to predict at least one biomarker. At step 880 training a neural network 1302 based upon the specific weights for each BMCD, the trained neural network 1302 configured to predict at least one biomarker, wherein the training of the neural network 1302 is accomplished by differently weighting weights of the BMCD that is used to train the neural network 1302. At step 890 deploying the trained neural network 1302; receiving biometric signal data 120 for a patient; generate corresponding body morphology composition data 1306 and apply the trained specific weights to the trained neural network 1302; predicting at least two biomarkers in response to applying the trained specific weights 1308 to the body morphology composition data 1306.

In some other examples, the body morphology composition data 1306 [BMCD] is at least one of: patient age, sex, size, weight, body fat, demographic data, muscle mass, heart, lung, liver, spleen kidney, pancreas, prostate, breast, brain organ size, and bone density. The plurality of biomarkers associated with the body morphology composition data 1306 [BMCD] is at least one of: patient conditions, comorbidities, biomarkers, ICD 9 or ICD 10 codes (international classification of disease)], heart disease, diabetes, obesity and cancer. Other examples are possible.

According to another embodiment, pre-processing the biometric signal data 350 may be performed to generate unique biometric datasets before applying the biometric signal data to the trained neural network.

In other examples, the weighting assigns the trained specific weights for each BMCD. For example, a first weight of a first body morphology composition data is of greater importance than the second weight of a second body morphology composition data or a third weight of the third body morphology composition data. According to this embodiment, a class, group or subset of body morphology composition data has a higher correlation to a specific disease and thus is more relevant to an accurate or higher scoring Biomarker Predictive Information 340 and is further described with reference to FIGS. 22 and 36.

In other examples, the weighting assigns the first body morphology composition data a greater importance than the second body morphology composition data or the third body morphology composition data. According to this embodiment, a class, group or subset of body morphology composition data has a higher correlation to a specific disease and thus is more relevant to an accurate or higher scoring Biomarker Predictive Information 340 and is further described with reference to FIGS. 22 and 36.

It will be appreciated that as used herein the term “control circuit” refers broadly to any microcontroller, computer, laptop, medical device, tablet or processor-based device with processor, memory, and programmable input/output peripherals, which is generally designed to govern the operation of other components and devices. It is further understood to include common accompanying accessory devices, including memory, transceivers for communication with other components and devices, etc. These architectural options are well known and understood in the art and require no further description here.

It will be appreciated that the machine learning algorithms and/or models 904 may include an algorithm (implemented as a neural network) that produces a model. The model may be analyzed by other software at the data analytics engine 902 or by other control circuits, processes, processors, computers, humans, or other entities.

In other aspects, the machine learning algorithms 904 may include an algorithm (implemented as a neural network) that produces an output. The output may be some sort of mathematical representation such as a vector, graph, matrix, algorithm, code, or signal. The output may be transformed, converted, analyzed, or further processed by other software at the data analytics engine 902 or by other control circuits, processes, processors, computers, human personable, or other entities. The output may be in the form of a file (in any format with any type of contents including those mentioned above) and, as mentioned, may itself be considered a model. The output may be sent to other entities such as the data analytics engine 902 or smartphone control unit 918, where the output is further utilized, used, processed, refined, or interpreted and further actions taken based upon this usage, processing, refining, or interpreting.

Machine learning algorithms 904 may be of any structure or combination of or usage of structures such as files, data structures (within the files), code, pseudocode, graphs, vectors, weightings, equations, mathematical constructs, or algorithms to mention a few examples. These structures, in one example, are neural networks. In one specific example, machine learning algorithms 904 comprise a convolution neural network. As mentioned and in some examples, the machine learning algorithms 904 may be implemented at least in part by the data analytics engine 902 and memory (or other local, remote and/or virtual electronic processing devices and memories).

In aspects, the machine learning algorithms 904 are trained using training data and perform pattern recognition on the training data to build a model and/or train the algorithm. Examples of machine learning algorithms included artificial neural network/backpropagation-based algorithms, regression-based algorithms, and decision tree-based algorithms. Other examples of machine learning algorithms are possible. The machine learning algorithms 904 may be stored in the data analytics engine 902, database 906, combinations of these locations, or at any other location (e.g., some other electronic device or memory), or in any combination of locations).

In one example, the machine learning algorithm 904 is a neural network and the training of neural networks involves applying training data to the neural network. The training data may be based upon data from sensors of the smartphone being operated by a patient, data describing the biometric information 100 and patterns of the other patients with similar biomarkers, data describing smartphone parameters of smartphones operated by the other patients, and/or data concerning the components of the smartphone. Other examples are possible. In aspects, a neural network is stored in an electronic memory device and may include or represent different layers, weightings, algorithms, computer instructions, other structures, and/or data representing these or other features. It will be understood that the neural networks described herein are stored in electronic memory.

FIGS. 36 and 38 are flow charts for obtaining training data 3602, applying the training data 3604, determining the error 3606 and applying error to the network 3608 to retraining the weights. FIG. 34 is a block diagram including the neural network processing a machine learning algorithm 3406 is trained using an optimization algorithm and weights 3408, 3410, 3412 of the neural network are updated using a backpropagation of error algorithm or function at control circuit 3405. At step 3602 biometric detection device 3404, sends appropriate images to determine the error 3416 via the machine learning algorithm engine 3306, 3406. At step 3604, in this embodiment feedback information such as weights 3408, 3410, 3412 for tuning and adjustment are fed back to machine learning algorithm engine 3306.

At step 3606 the network with a given set of weights 3408, 3410, 3412 is used to make predictions and the error 3416 for those predictions is calculated. At step 3608, the error algorithm seeks to change the weights (see FIG. 38) so that the next evaluation reduces the error 3416, meaning the optimization algorithm is navigating down a gradient (or slope) of error. In examples, it is desired to minimize the error and a loss function is used to calculate an error or loss.

According to the embodiment in the flow chart of FIG. 38, at step 3808 a decision is made to determine if the biomarker disease correlation is less than a threshold correlation. If yes, at step 3820, the biomarker is discarded or is assigned a lower weight. If no, at step 3812 the biomarkers and associated weights have been optimized and assigned and the revised biomarkers and weights are ready for release in a production software version.

The machine learning algorithm 904 may be trained in a supervised or unsupervised way. Supervised algorithms select target or desired results, which are predicted from a given set of predictors (independent variables). Using these set of variables, a function or structure is generated that maps inputs to desired outputs. The training process continues until the algorithm achieves a desired level of accuracy on the training data. Examples of supervised learning algorithms include regression, decision tree, random forest, k-nearest neighbors (KNN), recursive portioning, self-organizing feature maps, model ensembles, and logistic regression approaches. In aspects, supervised learning can use labeled data.

In unsupervised learning, no targets are used. Instead, these approaches cluster populations into different groups according to patterns. Examples of unsupervised learning approaches include the Apriori algorithm and the K-means approach.

As mentioned, the training of the neural network may be made by labeled or unlabeled data. Labeling typically takes a set of unlabeled data and attaches or associate each piece of it with a tag or label. For example, a data label might indicate whether data is from a particular component, person, smartphone, time, medical, test data, or operation condition to mention a few examples. An operator makes judgments about a given piece of unlabeled data. Training may be accomplished using unlabeled data as well.

It will be appreciated that the structure of the neural network 3500, 1302 is physically changed or transformed as the neural network is trained. In examples, weightings used by the network are changed. In other words, the neural network as represented in electronic memory is physically changed.

As mentioned, the machine learning algorithms 904 may be of any structure or combination of or usage of structures such as files, data structures (within the files), computer code, pseudocode, graphs, vectors, weightings, mathematical equations, mathematical constructs, or algorithms to mention a few examples. These structures, in one example, are used to form neural networks. As mentioned, these structures may be stored in electronic memory. After the neural network is trained, a data analytics engine 902 or other electronic processing device may apply inputs to the neural network in the memory, and the processing device or data analytics engine 902 generates outputs from the neural network.

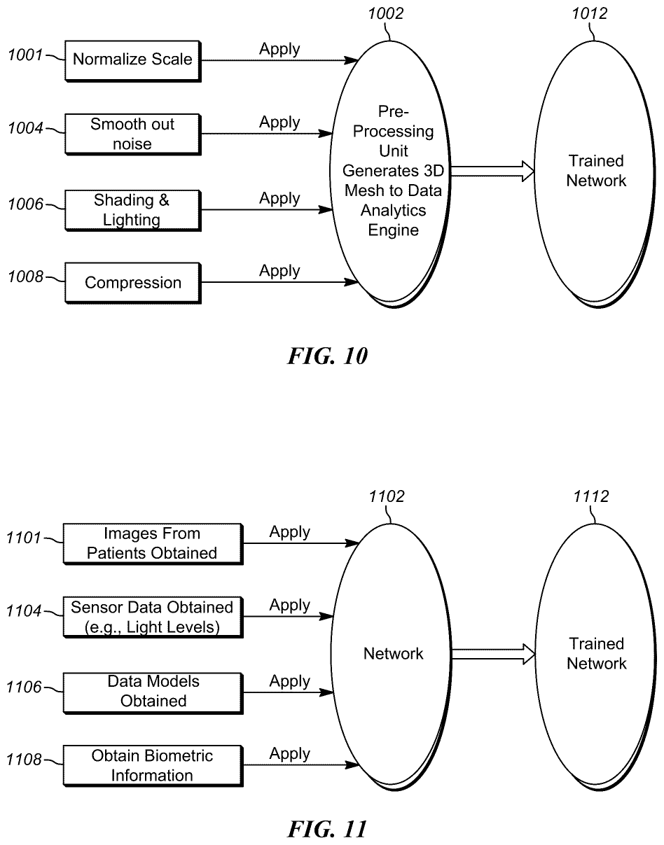

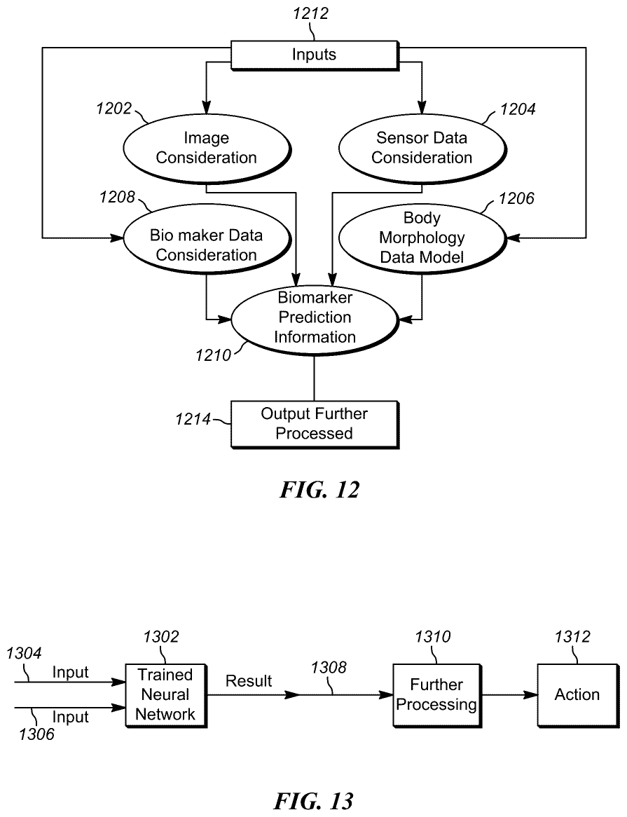

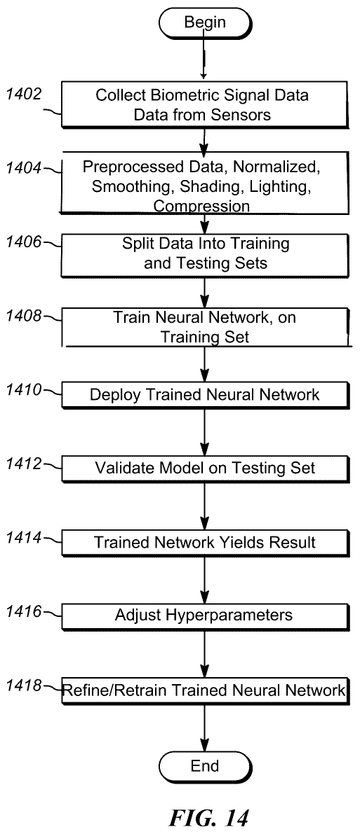

FIGS. 10-12, and 14 illustrate pre-processing and processing of data such as biometric data 100, 200 according to one embodiment. In some aspects, the techniques described herein relate to a disease prediction device configured to detect one or more biometric signal data 120, 250 and to predict disease comorbidity of one or more patients. The device includes: a biometric detection device 110, 210 configured to generate biometric signal data 120 of one or more patients; a trained neural network 1012, 1112, 1302. Images from patients 1101 are processed to: normalize 1001 the biometric signal data 150, 250 with at least one of: smoothing texture 1004, shading and lighting 1006; compression 1008; create a 3D (or 2D as previously described) volumetric mesh in response to the smoothed biometric signal data 120, 250, 350; render at least one view; identify a plurality of body morphology feature set data 190. Among other advantages, the processing of normalizing 1001, smoothing out noise 1004, shading and lighting, 1006 and compression 1008 result in a processed image highly suitable as biometric signal data 350 that is of a sufficiently small enough file size for a data analytics engine 330 to operate on a modest computing platform such as a smartphone, digital signal processor or medical device operated even on battery power.

A normalization processes generates biometric signal data 120, 250 to train the neural network. Normalization includes training regiments implemented such as contrast, smoothing/texturizing 1004, shading 1006 and generating an image file such as jpg to further compress and generate generates biometric signal data 120, 250. The generates biometric signal data 120, 250 is tested and validated. A feature set such as specific biomarker information 370 is identified as described above that results in a relatively small subset of data that requires significantly less processing than for example a 3D image file.

Among other advantages, 3D images native of MRI, XRAY and CAT images are projected and rendered into a 2D image to reduce the size of the file and to facilitate processing on a device of relatively modest computing power such as a handheld device. Further, the 2D image may provide suitable information for disease prediction by selecting specific biomarker information 370. The 3D images size has more information than is normally needed for predicting many diseases. For example, 3D native images typically provide the front, back and sides to create a 3D image and thus can provide internal images. However, the 2D image of the biometric signal data 120, 250, 350 can provide sufficient information about the body features to estimate for example fat distribution and then predict disease and or comorbidity. Inferences of for example the size and roundness of the belly may determine body fat, BMI level and thus predict disease such as diabetes, heart disease, lung disease, renal disease, vascular disease, cardiac arrythmias and stroke, dementia. For example, from this limited information, the body composition and body morphology can be inferred, including organ size such as the size of the liver spleen, heart and so forth. The correlation of these organ sizes with external body characteristics is verified by training the AI engine 3306, 3406, reviewing the results and determining the correlation with actual diagnosis. The large amounts of data based on these body morphology characteristics correlate to predicting diseases by the AI engine 3306, 3406. The images may be rendered by using polygons to create a mesh image model. According to one embodiment, the STL stereo lithographic file (other file formats, ply, jlb, obj, fbx, wave front object, film box, 3D studio or other suitable formats are possible) is processed to apply a light source, shading, smoothing and rendering. The 3D STL can be converted to PNG, JPG or any other suitable format. These image standards perform further image file compression by further processing of redundant data as is known in image processing. The 3D image may be projected to a 2D image by creating a rending window and a size by picking a rotational view.

Conventional techniques process 3D images however the sheer size of these files significantly increases processing loading making processing on portable devices impractical.

Normalization may further include selecting a normal color, display span or size and smoothing or texturing so that the biometric signal data 350 primarily identifies the selected biomarker information 370 characteristics while minimizing imaging artifacts. Smoothing for example can eliminate bumps and imperfections to minimize imaging artifacts. Blurring can also help smoothing.

Normalizing the brightness and contrast levels may be performed by determining the maximum and minimum normalized values, z score histotrophic, adaptive histogram, log distribution and power law, gamma transformation or any suitable normalization algorithm, process or transformation. For example, in a data set of images may be analyzed for a maximum and minimum values and thus total range of the value so that all the images in the data set are on the same scale. This ensures that the pixel values are in a certain range and are thus the images are normalized in an image size. Here are some common normalization techniques used for image processing:

### 1. Min-Max Normalization

This method scales the pixel values to a range, typically [0, 1] or [−1, 1].

\text{Normalized Pixel}=\frac{\text{Pixel}−\text{min}}{\text{max}−\text{min}} Formula:

This technique ensures that the minimum pixel value in the image becomes 0, and the maximum becomes 1 (or −1 and 1).

Use case: This is commonly used in image classification tasks with neural networks to ensure pixel values are within a certain range for better convergence during training.

### 2. Z-Score Normalization (Standardization)

Also known as mean normalization, this method adjusts the pixel values based on the image's mean and standard deviation.

\text{Normalized Pixel}=\frac{\text{Pixel}−\mu}{\sigma} Formula:

Where \mu is the mean pixel value, and \sigma is the standard deviation.

Use case: This method is preferred when working with datasets where pixel intensity distributions vary significantly. It centers the data around 0 with a standard deviation of 1.

### 3. Histogram Equalization

This technique adjusts the contrast of an image by redistributing the intensity values of the pixels so that the histogram (intensity distribution) becomes more uniform.

### Algorithm:

Compute the histogram of pixel intensities.

Calculate the cumulative distribution function (CDF) from the histogram.

Use the CDF to map the pixel values to a new intensity level.

Use case: Histogram equalization is useful for improving the contrast of images in low-contrast situations.

### 4. Adaptive Histogram Equalization (CLAHE)

A variant of histogram equalization, CLAHE applies the equalization process on small sections of the image, called tiles, rather than the whole image.

### Algorithm:

Divide the image into small tiles.

Apply histogram equalization to each tile.

Use bilinear interpolation to smooth the boundaries between tiles.

Use case: CLAHE is effective for enhancing local contrast while preventing over-amplification of noise.

### 5. Logarithmic Transformation

This technique enhances the darker regions of the image by compressing the higher intensity values using a logarithmic function.

\text{Transformed Pixel}=c\cdot\log(1+\text{Pixel}) Formula:

Where c is a scaling constant.

Use case: Log transformation is used to compress the dynamic range of images with large intensity differences, such as medical images or satellite images.

### 6. Power-Law (Gamma) Transformation

Gamma correction adjusts the pixel intensity based on a power-law transformation, which is effective for correcting brightness.

\text{Transformed Pixel}=c\cdot\text{Pixel}\hat{ }\gamma Formula:

Where c is a scaling constant, and \gamma is the gamma value. A gamma of 1 results in no change, while values less than 1 brighten the image and values greater than 1 darken it.

Use case: Gamma correction is frequently used in image display systems to account for the non-linear response of displays.

### 7. Mean Subtraction

In this technique, the mean of the image or the dataset is subtracted from each pixel value.

\text{Normalized Pixel}=\text{Pixel}−\mu Formula:

Where \mu is the mean of all pixels in the image or batch.

Use case: Mean subtraction is often used in deep learning models to center the data around 0, helping models converge faster.

8. Normalization with Predefined Statistics

For many pretrained models (e.g., those in PyTorch or TensorFlow), it's common to normalize images using predefined statistics (mean and standard deviation) from the ImageNet dataset or others.

### EXAMPLE

Pretrained ResNet models use the following normalization:

\text{Pixel}_{\text{norm}}=\frac{\text{Pixel}−\text{mean}}{\text{std dev}}

where the mean and standard deviation are typically [0.485, 0.456, 0.406] and [0.229, 0.224, 0.225] for RGB channels, respectively.

9. Contrast Stretching this is a Linear Normalization Technique where Pixel Values are Stretched to a New Range, Often the Full Range of the Data Type (e.g., 0-255 for 8-Bit Images).

\text{New Pixel}=\frac{\text{Pixel}−\text{min}}{\text{max}−\text{min}}\times (\text{new max}−\text{new min})+\text{new min} Formula:

Use case: Contrast stretching enhances the visibility of features in low-contrast images by using the entire dynamic range.

These normalization techniques and other suitable techniques are applied in many computer vision tasks to preprocess images, making them more consistent for algorithms and models, and improving performance across diverse datasets.

According to one embodiment, to render at least one view comprises at least one of: render an image with a 2D or 3D volumetric x-y mesh or wire frame corresponding with relative coordinates, creating a stereo lithograph file, a convolution smoother, and generate a 3D volumetric mesh to generate multiple views.

FIG. 37 is a flowchart for mapping biomarkers to disease predictions. At step 3702, gather and or generate body morphology composition data 1306 [BMCD] associated with the 3D (or 2D as described) mesh feature set for each 3D (or 2D as described) mesh data model. At step 3704 identify a patient and type for a plurality of biomarkers associated with the BMCD; At step 3708 generate and or map trained specific weights for each BMCD to predict at least one biomarker 1210.

It will also be appreciated that multiple machine learning algorithms 904 can be utilized. FIG. 35 is a diagram of an exemplary neural network 3500 having multiple layers of nodes or neurons. For example, multiple neural networks can be used with each neural network assigned to a patient. On the other hand, a single neural network that models all patients may be utilized. In other examples, separate and multiple neural networks may each be assigned to or make predictions concerning a particular geographical location, a particular smartphone component, particular intended actions (suggestions as to smartphone services, predictions as to smartphone parts, predictions that are used to control signals). The neural networks can be deployed at a central location, multiple central locations, at medical facilities, the cloud, at the smartphones, or combinations of these locations. It will be appreciated that the neural networks may be coupled together used any appropriate electronic communication network structure.

As mentioned, and in one specific example, the machine learning models 904 are neural networks 3500. The neural network(s) 3500 can have various layers and each of the layers performs one or more specific functions. In some aspects, these layers form a graph structure with vectors or matrices of weights with specific values. For instance, an input layer 3502-3508 receives input signals or data and transfers this information to the next layer 3510-3528 and may be hidden. One or more other layers perform calculations or make determinations on or involving the data. An output layer 3530-3534 transmits the result of the calculations or determinations. If the network is a convolution neural network (CNN), one or multiple convolutional layers are included in the network structure. In aspects, the convolutional layers apply a convolutional function on the input before transferring it to the next layer.

In other aspects, the neural networks include neurons, which are interconnected by connections or edges. In some examples, the neurons are formed into layers. Different layers may perform different transformations on their inputs. Signals travel through the neural network from the input layer, through other layers, and then through the output layer.

Each connection transmits a signal to other neurons. A neuron receives a signal then processes it and can signal neurons connected to it. In examples, the signal at a connection is a number (e.g., a real number). The output of each neuron is computed by a function of the sum of its inputs. Neurons and edges may have a weight that changes as the neural network is trained. In aspects, the weight increases or decreases the strength of the signal at a connection.

It will be appreciated that neural networks are one example of machine learning algorithms. Other examples include linear regression, logistic regression, decision tree, Bayer, and random forest algorithms. Still other examples are possible. These algorithms can be substituted for the neural networks described herein.

Neural networks and Artificial Intelligent (AI) systems generally train their AI engine at least via trial and error. The error can be measured based on patient or buyer satisfaction, advertising revenue, successful transactions completed withing a product or market or any suitable performance indicator. For example, if the performance indicator indicates a high degree of success then the system 900 may not need training or may be protected from potentially harmful training. If, however, the performance indicator indicates a low degree of success, then self-training and improvements can be performed. For example, scenarios of selected training data can be parsed and analyzed to identify faulty or poorly correlated training data. Scenarios that show removal of a subset of data result in a higher degree of success then the system 900 may revise the data accordingly.

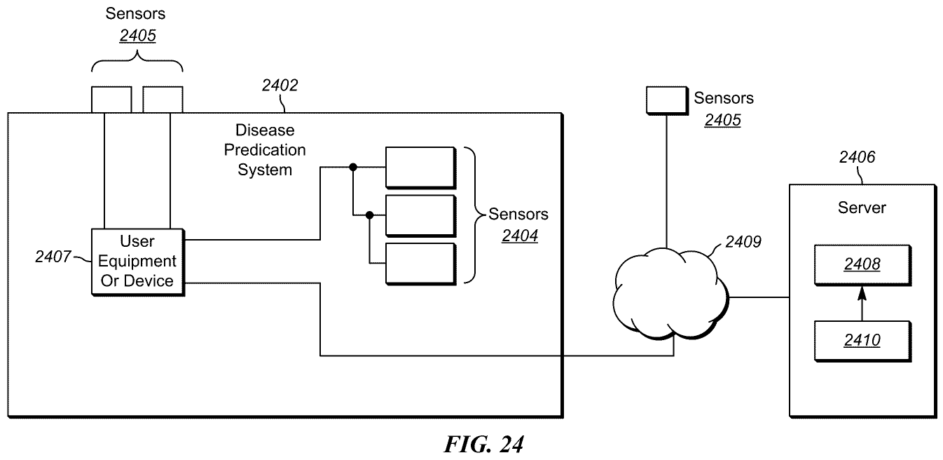

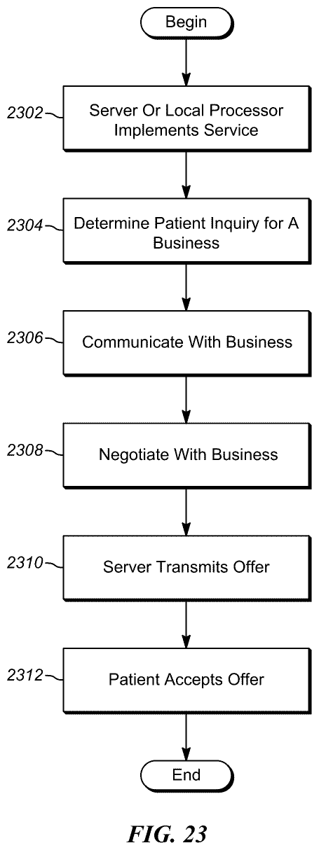

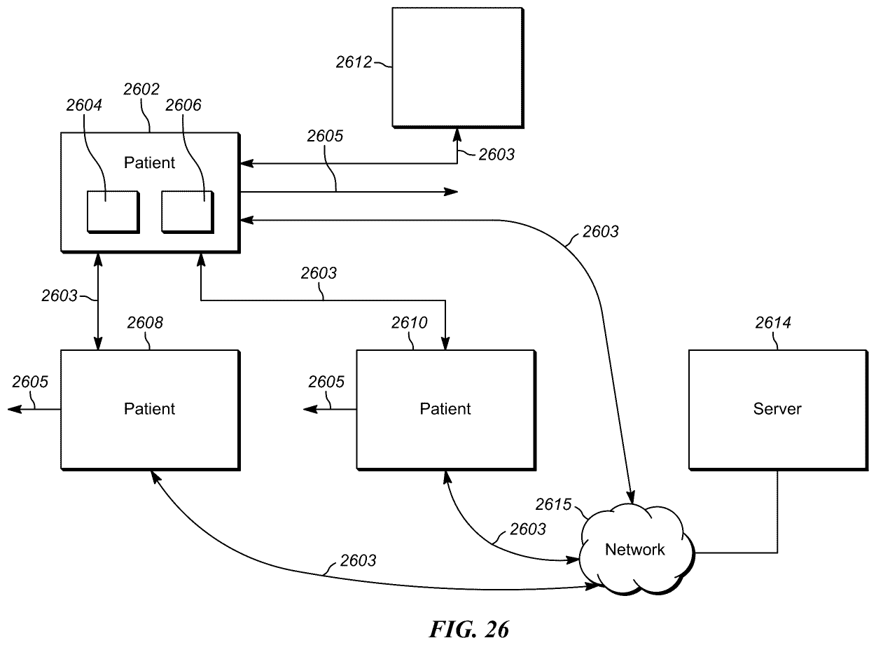

FIGS. 24, 25, 26 and 30 are block diagrams of a disease prediction system 2402 with one or more servers 2408 in a network 2409 in communication server 2406. Server 2406 may process data via an AI engine 2410 and blockchain engine 2408, sensors 2405 such as MRI and CAT scanners as well as from sensors 2404, 2405 from user equipment 2407. According to this embodiment the images are sent to servers 208 for processing as described so the appropriate prediction information is sent to the user equipment 2407.

FIG. 27 is a flowchart for a disease prediction business plan according to one embodiment. The business operations may be implemented and or facilitated for example in disease prediction offerings and sales device 3016. At step 2702, entities such as medical service providers, medical insurance providers, health fitness device makers and service providers or any suitable entity pays a fee for disease prediction services. At steps 2704, and 2706 the disease prediction information is generated. At step 2708 the medical service entity offers the service and at 2710 offers the disease prediction and prevention information to patients in consideration of payment or sales by for example disease prediction offerings and sales device 3016. At step 2712 both the model and the business plan and or payment model may be tuned, via the data analytics engine or via any suitable AI engine. Secured, verified, and or secured patient information such as biometric signal data 120 may be required for communication in the network 2409 between resources by one or more blockchain engines 2408 in a blockchain network 3005. For example, the disease prediction system 10 includes user equipment 3011, 3012 3013, 3014 at various smartphones, devices within smartphones, and an optional central server 2406 may be nodes in this network 2409. A blockchain is a data structure that stories a list of transactions and can be thought of as a distributed electronic ledger that records transactions between source identifier(s) and destination identifiers(s). The transactions are bundled into blocks and every block (except for the first block) refers to or is linked to a prior block in the blockchain. Computer resources or nodes maintain the blockchain and cryptographically validate each new block and the transactions contained in the corresponding bloc. Such a validation process includes computationally solving a resource intensive problem that is also easy to verify, secure, authenticate and is itself a proof of work, such as a hash function. Security may further be provided for example for the testing information as appropriate to maintain the confidential and proprietary nature of the testing information. In another embodiment, communication may implement cryptography and secure communications such as OpenSSL, via a commercial grade cryptography, secure communication, encryption software and toolkits. OpenSSL is a free and open-source software for general-purpose cryptography and secure communication.

FIG. 31 is a block diagram of a patient's electronic device 3100 according to one embodiment. The patient's electronic device 3100 includes a transmitter/receiver 3102, a machine learning algorithm(s) 3104 and an electronic memory device 3108. The patient information such as biometric signal data 120 may be stored on a distributed ledger and/or a blockchain. In some embodiments, a patient's key (e.g., encryption key or public key) may be received by, for example, a control circuit 3106 processor, computer 3106, memory 3108 and/or blockchain administrator from, for example, a patient's electronic device 3100 (e.g., smart phone or computer, laptop, medical device, tablet). The key may be verified and, responsively to the verification, an outcome measurement device (OMD) (e.g., a medical questionnaire, medical test results, EKG, CPAP) may be provided to a patient so that, for example, it may be displayed on his or her personal electronic device 3100.

The blockchain may be public, private, or some combination thereof. In some embodiments, the set of biomarker information 270, 370 diagnostic biomarker predictive information 140, 240, 340 and/or body morphology composition data 1306 for a patient may be associated with the patient's electronic medical record (prior to, or following, the receipt, encryption and/or broadcasting. Additionally, or alternatively, in some instances, the set of responses may be digitally signed. This may occur prior to packaging the set of biomarkers 270, diagnosis and/or body morphology composition data 1306 and if so, the digitally signed and packaged set of data may be securely broadcast to the blockchain.

Even when disease prediction system 10 is privately operated and/or distributed ledger and/or blockchain is private, interaction between disease prediction system 10 components such as the user device 2407 and trained neural network, server 2614, 3005, computers and/or distributed ledger and/or blockchain may be user device agnostic in that no specific requirements for the user device (other than ability to communicate with server) may be required. Thus, information may be able to pass between user device 2407, trained neural network, server 2614, 3005, computers and/or distributed ledger and/or blockchain regardless of software or hardware constraints.

Patient information may be stored in patient account database and/or EMR database and a link to patient information may be stored in distributed ledger and/or blockchain. In these embodiments, a request for patient information may be communicated to server by user device or communicated to server 3008 by treatment providers/facility device 3007. After verifying that the respective user of user device 3002, 3004, 3006 and/or treatment provider/facility device it is authorized to access the requested patient information, server or server 3008 may include within these devices and access distributed ledger and/or blockchain to retrieve the requested patient information. If the requested patient information is stored on distributed ledger and/or blockchain, the request of patient information may be extracted from distributed ledger and/or blockchain and provided to user device or treatment provider/facility device.

Distributed ledger and/or blockchain may store a link to the requested patient information and not the patient information itself. When a request for patient information is received by server such as for example disease prediction offerings and sales device 3016 in consideration of payment or sales, the server may query distributed ledger and/or blockchain for the information and/or a link to the patient information. When distributed ledger and/or blockchain includes a link to the patient information, this link may be provided to and/or extracted by server. Server 3008 and or disease prediction offerings and sales device 3016 may then provide the link to the requesting device (i.e., user device of treatment provider/facility device) and/or may use the link to retrieve the information associated therewith from the patient account database or patient EMR database and may then provide the retrieved information to the requesting device. Storing a link to patient information (as opposed to the patient information itself) on distributed ledger and/or blockchain may serve to, for example, preserve storage space on the distributed ledger and/or blockchain and/or increase privacy for the patient information. In some cases, security of the patient information may be augmented via one or more security protocols associated with the link and/or activation thereof.

In some embodiments, an indication of fulfillment of the security measures may be packaged for storage on the blockchain. This packaging may include association of biomarkers, diagnosis and/or body morphology composition data 1306 or patient information and/or a patient identifier. Then, the packaged indication may be broadcast to the blockchain. In some embodiments, the patient may be provided with an indication that the condition has been fulfilled in the form of, for example, a message communicated to his or her electronic device.

In still other examples and as described herein, the user equipment 1700 communicates (directly or indirectly) with other smartphones. Data may be received and/or exchanged with these smartphones, in one example. This data may be used by the artificial intelligence algorithms to make more accurate or effective recommendations, predictions, or suggested actions.

Once the pre-processing is complete, the pre-processed data can be applied to the data analytics engine 902. Various algorithms and machine learning approaches can be used to accomplish the pre-processing of data. For example, various types of data compression algorithms can be utilized to compress data. In other aspects, training data can also be pre-processed before being used to train a machine learning algorithm such as a neural network,

Several benefits and advantages that may be provided by the present application have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations that follow those terms. Accordingly, a system, method, or other embodiment that comprises a set of elements is not limited to only those elements and may include other elements not expressly listed or inherent to the claimed embodiment.

What is claimed is:

1. A diagnostic disease prediction system configured to predict disease comorbidity of one or more patients in response to biometric signal data, comprising:
a biometric detection device configured to generate biometric signal data of one or more patients;
an electronic memory that includes data representing a trained neural network that has been trained to produce biomarker prediction information used in diagnosis, the trained neural network being made according to the biometric signal data, and biomarker information being personalized to an individual patient as defined by the biometric signal data to:
create normalized biometric signal data in response to processing the biometric signal data with at least one of: smoothing texture, shading and lighting;
create a 3D volumetric mesh data model in response to the normalized biometric signal data;
render at least one view of the 3D volumetric mesh data model;
identify a plurality of body morphology feature set data;
generate body morphology composition data [BMCD] associated with 3D volumetric mesh feature set data for each 3D volumetric mesh data model;
identify a plurality of biomarkers associated with the body morphology composition data [BMCD]; and
generate trained specific weights for each body morphology composition data [BMCD] to predict at least one biomarker;
a data analytics engine coupled to the trained neural network in the electronic memory;
wherein the trained neural network is subsequently deployed and the data analytics engine is configured to subsequently:
receive the biometric signal data for a patient, generate corresponding body morphology composition data for the patient and apply the trained specific weights to the trained neural network;
predict at least two biomarkers in response to applying the trained specific weights to the body morphology composition data; and
generate disease prediction information in response to predicting at least two biomarkers.

2. The diagnostic disease prediction system of claim 1, further including retraining the trained neural network based on the trained specific weights for each body morphology composition data [BMCD] to predict at least one biomarker and generate revised biomarker prediction information.

3. The diagnostic disease prediction system of claim 1, wherein at least one biomarker information being personalized to the individual patient comprises displaying at least one of: biomarker information and disease prediction information to the patient using a smart phone, personal computer, laptop, medical device or tablet.

4. The diagnostic disease prediction system of claim 1, wherein to create normalized biometric signal data further comprises:
projecting the biometric signal data from a 3D image onto a 2D plane to generate 2D biometric image data;
processing the 2D biometric image data and reducing a 2D biometric image data size for processing on a battery operated device according to at least one of:
smoothing or filtering to generate textured data;
coloring to generate colored data;
lighting to generate light adjusted data; and
shadowing the 2D biometric image data.

5. The diagnostic disease prediction system of claim 1, wherein the trained specific weights for each BMCD comprises a first weight of a first BMCD is greater than a second weight of a second BMCD and a third weight of a third BMCD.

6. The diagnostic disease prediction system of claim 1, wherein the biometric signal data from a patient is data according to at least one of: 3D image, 2D image, lidar, X-ray, MRI, CAT, medical history, EKG, CPAP information and medical test results.

7. The diagnostic disease prediction system of claim 1, wherein the body morphology composition data [BMCD] is data according to at least one of: patient age, sex, size, weight, body fat, demographic data, muscle mass, heart, lung, liver, spleen kidney, brain, pancreas, prostate, breast, organ size, and bone density.

8. The diagnostic disease prediction system of claim 1, wherein the trained neural network is deployed at a remote server location.

9. The diagnostic disease prediction system of claim 1, wherein the plurality of biomarkers associated with the body morphology composition data [BMCD] is at least one of: patient conditions, comorbidities, biomarkers, ICD 9 or ICD 10 codes (international classification of disease), heart disease, diabetes, obesity and cancer.

10. The diagnostic disease prediction system of claim 1, wherein the biometric detection device comprises one or more of radar, LIDAR sensors, cameras, ultrasonic sensors, MRI, X-Ray, CAT, mobile phone camera, and environmental sensors.

11. The diagnostic disease prediction system of claim 1, further comprising pre-processing the biometric signal data and reducing a biometric signal data size for processing on a battery operated device, to generate unique biometric datasets before applying the biometric signal data to the trained neural network.

12. The diagnostic disease prediction system of claim 1, wherein to render at least one view comprises at least one of: render an image with a 2D or 3D volumetric x-y mesh or wire frame corresponding with relative coordinates, creating a stereo lithograph file, a convolution smoother, and generate a 3D volumetric mesh to generate multiple views.

13. The diagnostic disease prediction system of claim 1 wherein at least one of: the biometric signal data, the biometric signal data and the body morphology composition data [BMCD] is processed, verified, secured, authenticated or stored on a blockchain.

14. A method for diagnostic disease comorbidity prediction and treatment, the method comprising:
obtaining biometric signal data of one or more patients;
smoothing the biometric signal data with at least one of: shading and lighting;
creating a 3D volumetric mesh data model in response to smoothing the biometric signal data;
rendering at least one view of the 3D volumetric mesh data model;
identifying a plurality of body morphology feature set data;
generating body morphology composition data [BMCD] associated with 3D volumetric mesh feature set data for each 3D volumetric mesh data model;
identifying a plurality of biomarkers associated with the body morphology composition data [BMCD];
generating trained specific weights for each body morphology composition data [BMCD] to predict at least one biomarker;
training a neural network based upon the trained specific weights for each body morphology composition data [BMCD], the trained neural network configured to predict at least one biomarker, wherein the training of the neural network is accomplished by differently weighting weights of the body morphology composition data [BMCD] that is used to train the neural network;
deploying the trained neural network;
receiving biometric signal data for a patient;
generate corresponding body morphology composition data and apply the trained specific weights to the trained neural network; and
predicting at least two biomarkers in response to applying the trained specific weights to the body morphology composition data.

15. The method of claim 14, further comprising retraining the trained neural network based on the trained specific weights for each body morphology composition data [BMCD] to predict at least one biomarker.

16. The method of claim 14, wherein the at least one biomarker is personalized to the patient comprises displaying the at least two biomarkers to the patient using a smart phone, personal computer, laptop, tablet, medical device or remote server.

17. The method of claim 14, wherein normalizing the biometric signal data further comprises at least one of:
projecting a 3D image onto a 2D plane;
smoothing or filtering image texture;
coloring;
lighting; and
shadowing the biometric signal data.

18. The method of claim 14, wherein the biometric signal data from a patient is at least one of: 3D image, 2D image, lidar, X-ray, MRI, CAT data.

19. The method of claim 14, wherein the body morphology composition data [BMCD] is at least one of: patient age, sex, size, weight, body fat, demographic data, muscle mass, heart, lung, liver, spleen kidney, organ size, and bone density.

20. The method of claim 14, wherein the at least two biomarkers associated with the body morphology composition data [BMCD] is at least one of: patient conditions, comorbidities, biomarkers, ICD 9 or ICD 10 codes (international classification of disease).

21. The method of claim 14, wherein the neural network is deployed at a central location.

22. The method of claim 14, wherein the biometric signal data is generated from at least of: radar, LIDAR sensors, cameras, ultrasonic sensors, MRI, X-Ray, CAT, mobile phone camera, and environmental sensors.

23. The method of claim 14, further comprising pre-processing the biometric signal data before applying the biometric signal data to the trained neural network.

24. The method of claim 23, wherein to render at least one view comprises at least one of: to render the biometric signal data into an image with a 2D or 3D volumetric x-y mesh or wire frame corresponding with relative coordinates, creating a stereo lithograph file, a convolution smoother and generating a 3D volumetric mesh to generate multiple views.

25. The method of claim 14 further including storing at least one of: the biometric signal data, the biometric signal data and the body morphology composition data [BMCD] on a blockchain.

26. A diagnostic disease prediction device configured to detect one or more biometric signal data and to predict disease comorbidity of one or more patients, comprising:
a biometric detection device configured to generate biometric signal data of one or more patients;
a trained neural network to:
normalize the biometric signal data in response to processing the biometric signal data with at least one of: smoothing texture, shading and lighting;
create a 3D volumetric mesh data model in response to normalize the biometric signal data;
render at least one view of the 3D volumetric mesh data model;
identify a plurality of body morphology feature set data;
generate body morphology composition data [BMCD] associated with 3D volumetric mesh feature set data for each 3D mesh data model;
identify a plurality of biomarkers associated with the body morphology composition data;
generate trained specific weights for each BMCD to predict at least one biomarker;
a data analytics engine coupled to the trained neural network;
wherein the trained neural network is subsequently deployed and the data analytics engine is configured to subsequently:
receive biometric signal data, generate corresponding body morphology composition data for a patient and apply the trained specific weights to the trained neural network;
predict at least one biomarker in response to applying the trained specific weights to the body morphology composition data; and
generate disease prediction information in response to predicting at least two biomarkers

27. The diagnostic disease prediction device of claim 26, wherein the trained neural network is retrained to reflect new biometric signal data.

28. The diagnostic disease prediction device of claim 26, including displaying the disease prediction information to the patient using a smart phone, personal computer, laptop, or tablet and in response receiving payment.

29. The diagnostic disease prediction device of claim 26, wherein the trained neural network is deployed at a remote server location.

30. The diagnostic disease prediction device of claim 26, wherein the biometric detection device sends biometric signal data to the trained neural network deployed at a remote server location and in response the remote server location sends the disease prediction information to the disease prediction device to display the disease prediction information to the patient.

31. The diagnostic disease prediction device of claim 26, wherein at least one of: a biometric signal data size, a normalized biometric signal data size, a 3D volumetric mesh data model size, a rendered view data size, a body morphology composition data size, a biomarker data size, and a trained specific weight data size, is reduced in data size for generating disease prediction information on a battery operated device.
